SEQUENCE LISTING (1 sequence)

SEQ ID NO: 1 - protein, 191 aa, Homo sapiens [sequence omitted]

(19) United States
(12) Patent Application Publication
(10) Pub. No.: US 2012/0116027 A1
(43) Pub. Date: May 10, 2012

(54) PROCESS FOR THE PURIFICATION OF HUMAN GROWTH HORMONE POLYPEPTIDES USING AFFINITY RESINS COMPRISING SPECIFIC LIGANDS

(76) Inventors: Jakob Ewald Rasmussen, Kobenhavn N (DK); Phaedria Marie St. Hilaire, Kobenhavn NV (DK); Michael Roice, Frederiksberg (DK); Christine Bruun Schioedt, Bronshoj (DK); Knud Jørgen Jensen, Kobenhavn N (DK)

(21) Appl. No.: 13/128,770

(22) PCT Filed: Nov. 13, 2009
(86) PCT No.: PCT/EP09/65143

§ 371 Date: Jan. 3, 2012

Related U.S. Application Data
(60) Provisional application No. 61/116,062, filed on Nov. 19, 2008.

(30) Foreign Application Priority Data
Nov. 13, 2008 (EP) 08169014.1

(51) Int. Cl. C07K 5/083; C08F 8/30; C07K 5/097; C07K 5/09; C07K 1/22; C07K 5/087

(52) U.S. Cl. 525/333.6; 530/399; 530/322; 530/331

(57) ABSTRACT

The present invention relates to a novel process for the purification of growth hormone polypeptides, e.g. recombinant human Growth Hormone. The process utilizes an affinity resin comprising a solid phase material having immobilized thereto one or more low-molecular weight synthetic ligands. The affinity resins enable the separation of Growth Hormone from closely related proteins.

### FIELD OF THE INVENTION

The present invention relates to a novel process for the purification of growth hormone polypeptides, e.g. recombinant human Growth Hormone. The process utilizes an affinity resin comprising a solid phase material having immobilized thereto one or more low-molecular weight synthetic ligands. The affinity resins enable the separation of Growth Hormone from closely related proteins.

### BACKGROUND OF THE INVENTION

Therapeutic proteins are produced in living cells and must be purified from a complex mixture of proteins and other biological species before use in a patient. This separation can be very laborious and costly. A number of chromatography materials have been described for use in such separation.

Affinity chromatography enables selectively and reversibly adsorbing biological substances, such as proteins, to a complementary binding substance, such as an affinity ligand immobilised on a solid phase material, usually a porous, polymer matrix, packed in an affinity column. A suitable ligand is covalently attached to the solid phase materials directly or by means of a linker. A sample containing biological substances having affinity for the ligand can be brought into contact with the affinity ligand covalently immobilised to the solid phase material under suitable binding conditions which promote a specific binding between the ligand and the biological substance having an affinity for the ligand. The affinity column can subsequently be washed with a buffer to remove unbound material, and in a further step, the biological substances having affinity for the ligand can be eluted and obtained in a purified or even in an isolated form. Accordingly, the ligand should preferably exhibit specific and reversible binding characteristics to the biological substance, which it is the aim to purify or isolate.

A number of chromatographic materials and processes have been described for the purification of human Growth Hormone (hGH).

U.S. Pat. No. 5,047,333 discloses a method for purifying human Growth Hormone using ion exchange chromatography or immobilized metal affinity chromatography.

U.S. Pat. No. 4,332,717 discloses a method for purifying human Growth Hormone using hydrophobic interaction chromatography in which a water insoluble carrier having hydrophobic groups such as alkyl- or phenyl-groups is used as a chromatographic material.

The above two methods rely on traditional purification techniques exploiting the differences in physico-chemical properties of the compounds. These, however, often require several laborious purification steps consequently leading to lower yields and higher costs.

U.S. Pat. No. 6,117,996 discloses affinity ligand-matrix conjugates comprising a ligand with the general formula.

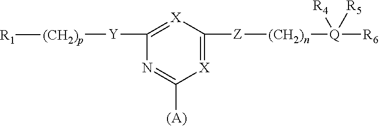

The ligand is attached to a support matrix in position (A), optionally through a spacer arm interposed between the matrix and ligand. The purification of proteinaceous materials such as e.g. immunoglobulins, insulins, Factor VII, or human Growth Hormone or analogues, derivatives and fragments thereof and precursors using such affinity ligand-matrix conjugates is also disclosed.

U.S. Pat. No. 5,760,187 discloses a method for the purification of human Growth Hormone using blue dyes such as Cibacron 3GA:

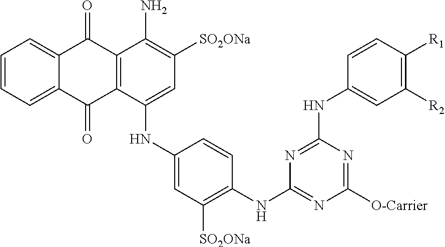

covalently attached to a support matrix. Blue dyes have long been used in affinity chromatographic purifications of adenyl-containing cofactor dependent enzymes such as trypsin or lactate dehydrogenase. However, elution of growth hormone from the carrier requires protein denaturing agents such as 6 M urea.

The chromatography materials described in the above prior art can indeed be used for purification of hGH. However, there is still a need for chromatography materials with high specificity towards hGH, and which materials comprises synthetic affinity ligands, and which materials further enables a purification process involving fewer steps and milder elution conditions.

Currently recombinant human Growth Hormone (rhGH) can be purified from the fermentation supernatant by different processes:

Conventional chromatography employing multiple chromatographic steps, such as by the methods disclosed in U.S. Pat. No. 5,268,277 and U.S. Pat. No. 5,633,352;

Affinity chromatography employing an affinity resin with protein ligands, e.g. as in U.S. Pat. No. 5,350,836; or

Affinity chromatography employing an affinity resin with synthetic small molecule ligands, e.g. as in U.S. Pat. No. 5,760,187.

Conventional chromatography involving multiple steps suffers from one or more of the following: low overall yield, high buffer consumption, long process time and large investment in process equipment, increased labour.

Affinity resins with protein ligands result in increased selectivity, higher yield, lower buffer consumption, and reduced investment in process equipment. However, affinity resins with protein ligands are very costly to manufacture as well as less chemically and conformationally stable compared to small synthetic ligands and inherently hold a risk that the purified rhGH be contaminated with protein fragments from the protein ligands or other biological matter from the production of the affinity resin with protein ligands.

Currently reported synthetic ligands for affinity chromatography of human Growth Hormone such as those disclosed in U.S. Pat. No. 5,760,187 require the use of strong protein denaturing agents to release the protein from the affinity resin.

Therefore there is a need for better affinity resins for the purification of recombinant human Growth Hormone (rhGH).

### SUMMARY OF THE INVENTION

The present invention provides a process for the purification of a Growth Hormone polypeptide using new affinity resins. The present invention provides for affinity resins comprising novel synthetic affinity ligands that are selective for Growth Hormone polypeptide, in particular human growth hormone (hGH), such as rhGH, and are not based on protein ligands and which are cheap and base stable.

### BRIEF DESCRIPTION OF THE DRAWINGS

FIG. 1. Average fluorescence values (arbitrary units) for AA1 (top), AA2 (middle), and CA (bottom).

FIG. 2. SDS page gels from selectivity testing of ligand 11 on Fractogel Amino (left) and ligand 6 on Fractogel Amino (right).

FIG. 3. Chromatogram showing a typical run using affinity resin with ligand L10.

FIG. 4. SDS-PAGE gel of a purification using affinity resin with ligand L10. Lane 1: Mark 12 marker; lane 2: flow-through; lane 3: eluate (affinity resin with ligand L10); lane 4: purified hGH (lacking amino extension); lane 5: micro-filtrated E. coli cell culture fluid.

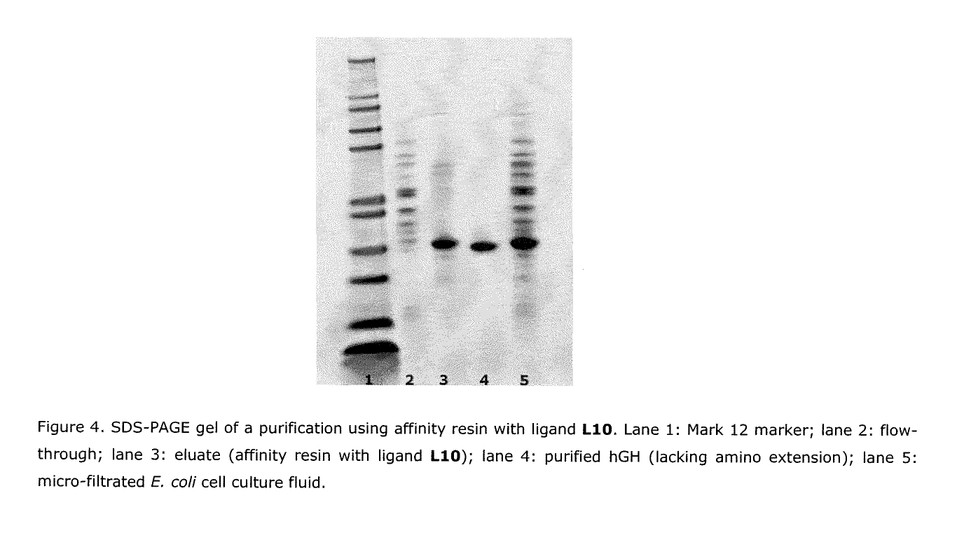

FIG. 5. Chromatogram of affinity purification of hGH from micro-filtrate from hGH harvest using ligand L10 to Fractogel Amino M.

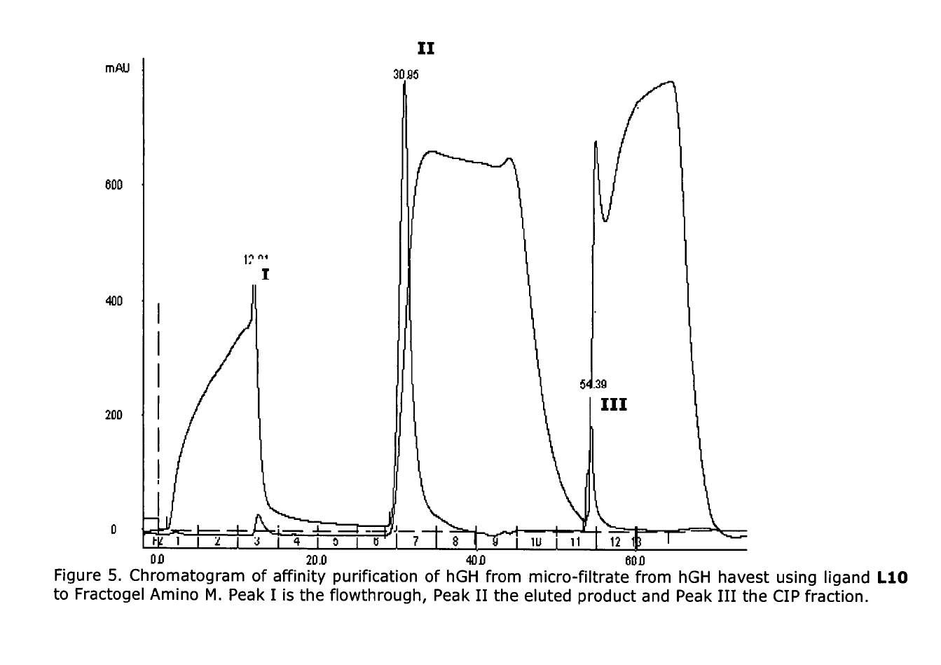

FIG. 6. SDS-PAGE of a purification using affinity resin with ligand L10. Lane 1 (from left): Protein marker Mark12 (Invitrogen); Lane 2: Flow-through (fractions 1+2+3 pooled, 10 μL sample on gel); Lane 3: Elution (fractions 6+7+8 pooled, 10 μL sample on gel); Lane 4: CIP (fractions 10+11+12 pooled, 10 μL sample on gel); Lane 5: Reference micro-filtrate sample (diluted ×50).

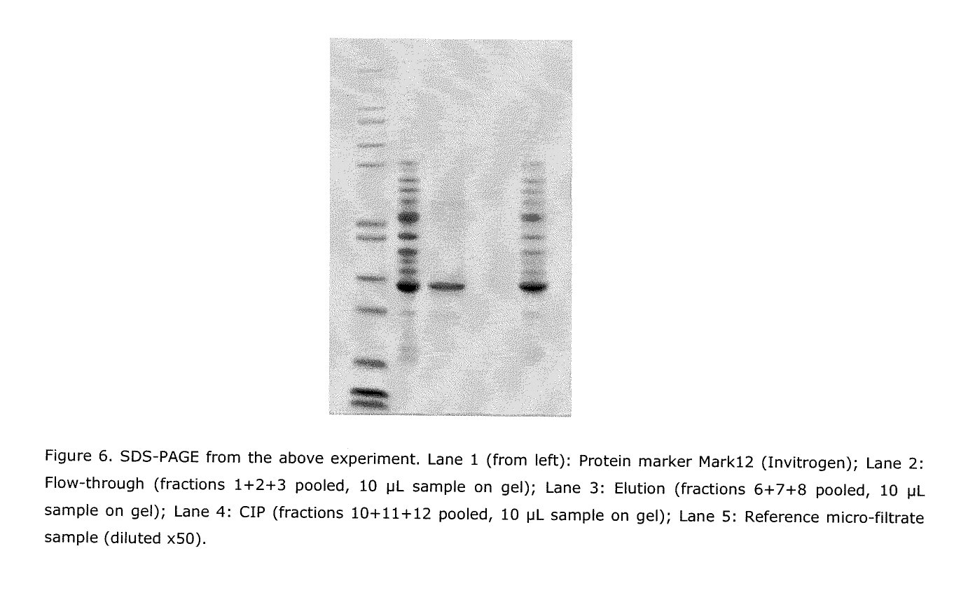

FIG. 7. HPLC of pooled fractions shown in Lane 3 in the SDS-PAGE. Purity 74%, hGH at Tᵣ=22 min.

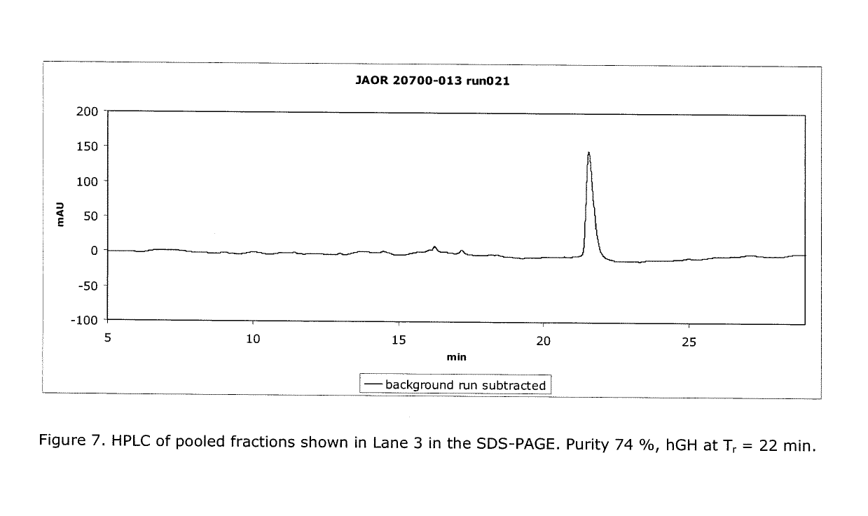

FIG. 8. Chromatogram obtained with load buffer: 50 mM BisTris at pH 6.25.

FIG. 9. SDS-PAGE of a purification using affinity resin with ligand L10. Lane 1 (from left): Protein marker Mark12 (Invitrogen); Lane 2: Flow-through (fractions 1+2+3 pooled, 10 μL sample on gel); Lane 3: Elution (fractions 6+7+8 pooled, 10 μL sample on gel); Lane 4: CIP (fractions 10+11+12 pooled, 10 μL sample on gel); Lane 5: Reference micro-filtrate sample diluted ×50)

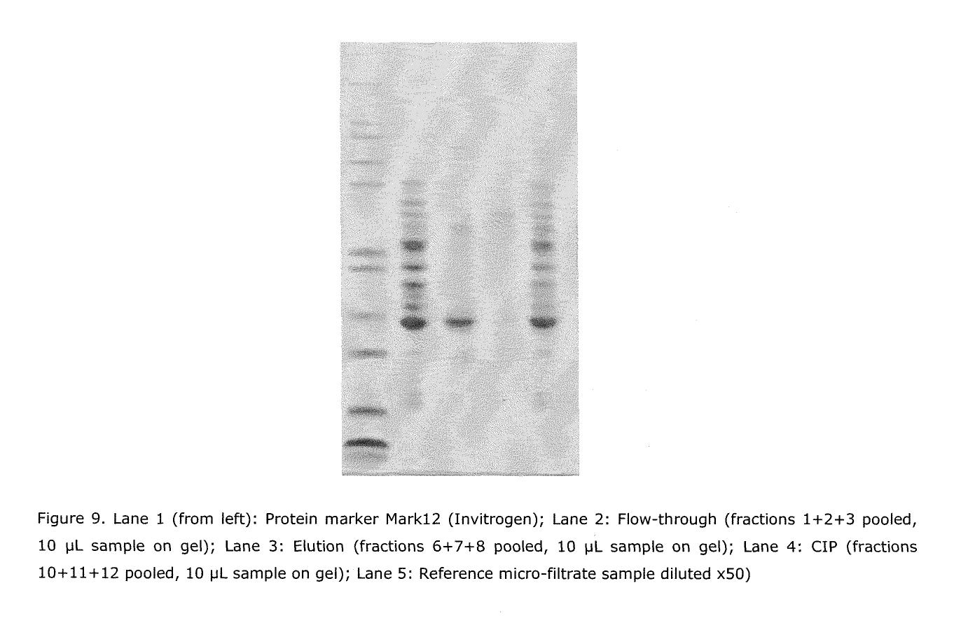

FIG. 10. HPLC chromatogram of pooled elution fractions (fractions 6-8), hGH at Tᵣ=21.5 min.

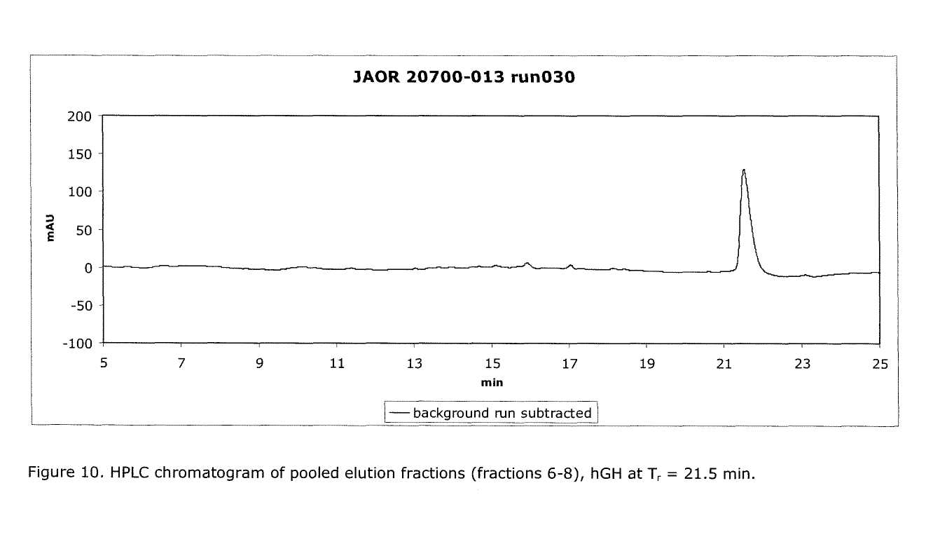

FIG. 11. Chromatogram from affinity purification of hGH using ligand L14 on Fractogel.

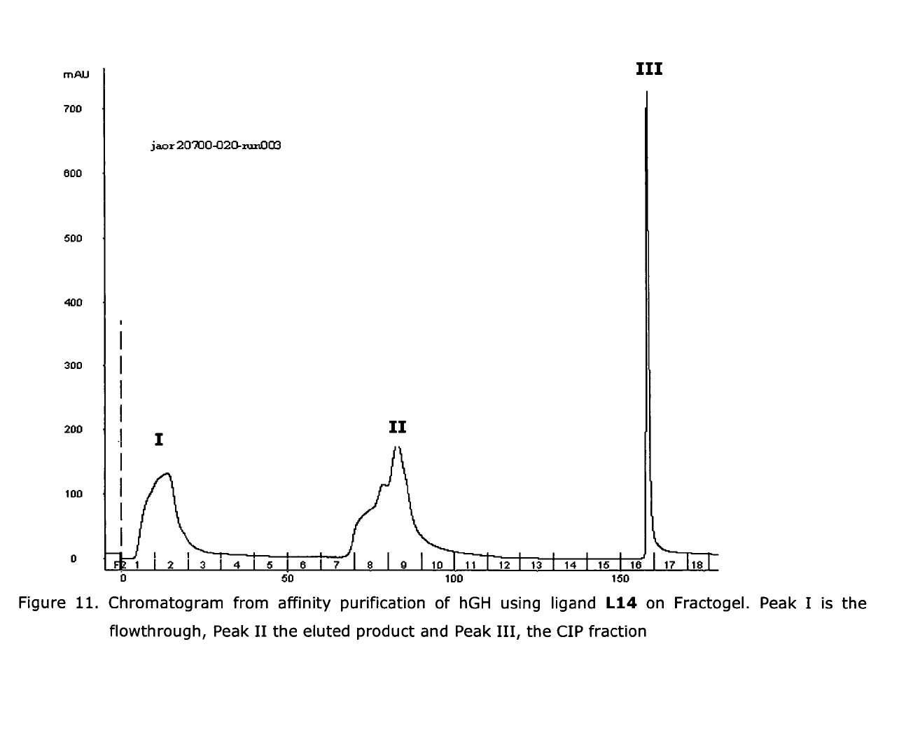

FIG. 12. SDS-PAGE of a purification using affinity resin with ligand L14. Gel analysis. Lane 1 (from left): Protein marker Mark12 (Invitrogen); Lane 2: Flow-through (fractions 1+2 pooled, 10 μL sample on gel); Lane 3: Elution (fraction 8, 10 μL sample on gel); Lane 4: Elution (fraction 9 (10 μL sample on gel)); Lane 5: Elution (fraction 7+8+9 pooled, 10 μL sample on gel); Lane 6: CIP (fractions 16+17 pooled, 10 μL sample on gel); Lane 7: hGH reference; Lane 5: Reference micro-filtrate sample diluted ×50).

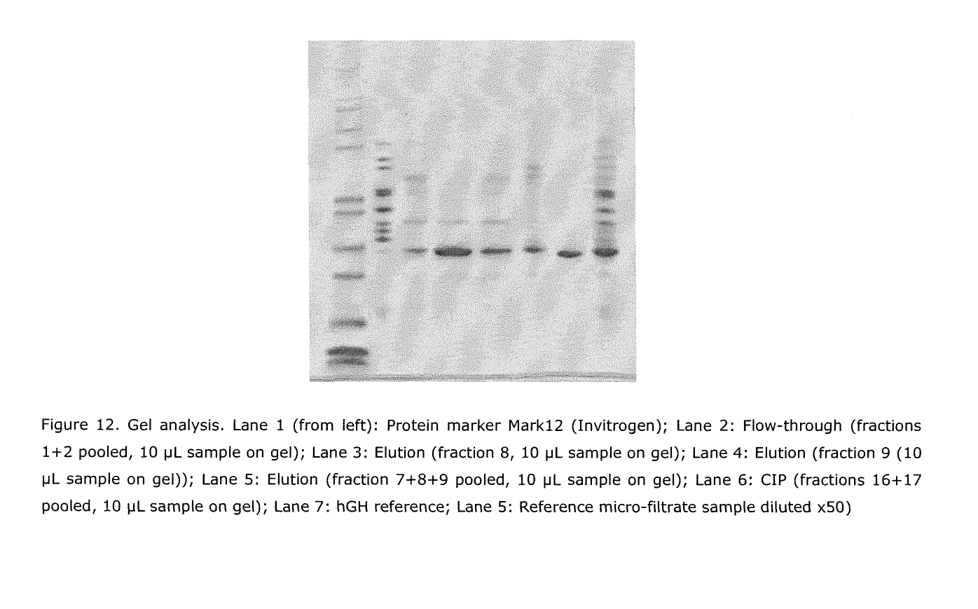

FIG. 13. HPLC chromatogram of elution of fraction 9.

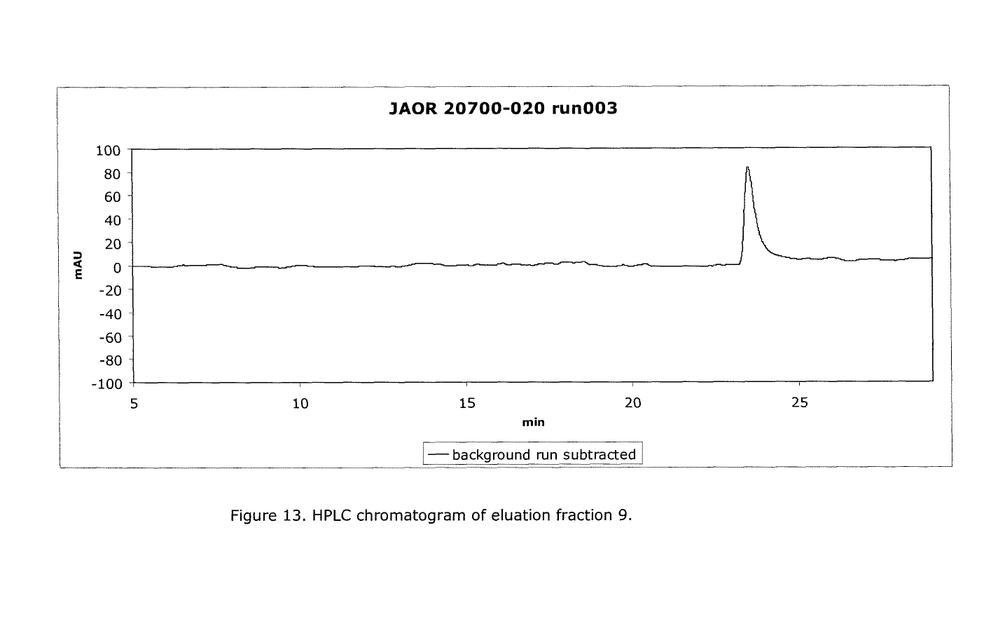

FIG. 14. Chromatogram from affinity purification of hGH using ligand L16 on Fractogel.

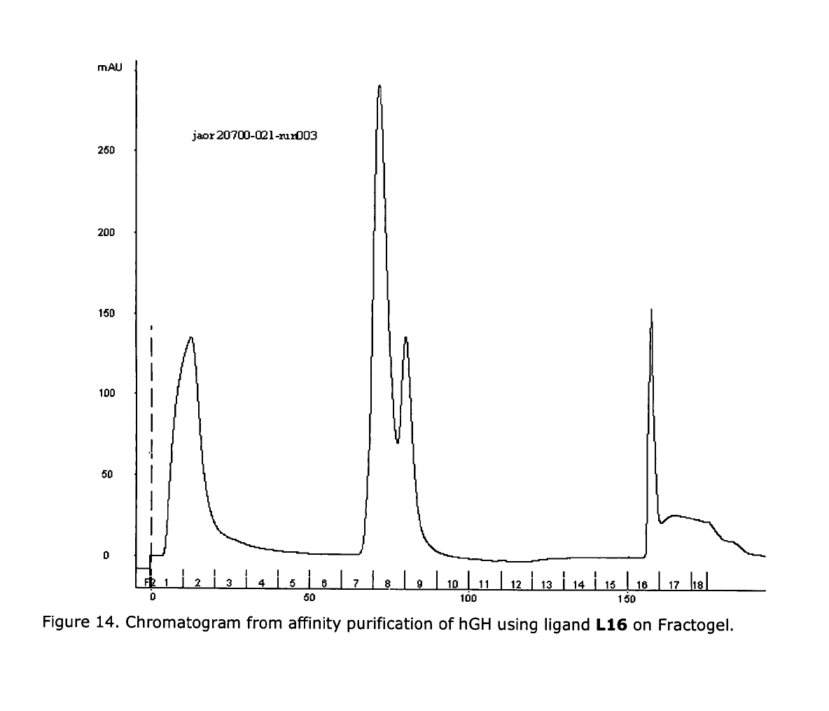

FIG. 15. SDS-PAGE of a purification using affinity resin with ligand L16. Lane 1: Protein marker Mark12 (Invitrogen); Lane 2: Flow-through (fractions 1+2 pooled, 10 μL sample on gel); Lane 3: Elution (fraction 7+8, 10 μL sample on gel); Lane 4: Elution (fraction 9, 10 μL sample on gel); Lane 5: Elution (fraction 7+8+9 pooled (10 μL sample on gel)); Lane 6: CIP (fractions 16+17 pooled, 10 μL sample on gel); Lane 7: hGH reference; Lane 5: Reference micro-filtrate sample diluted ×50)

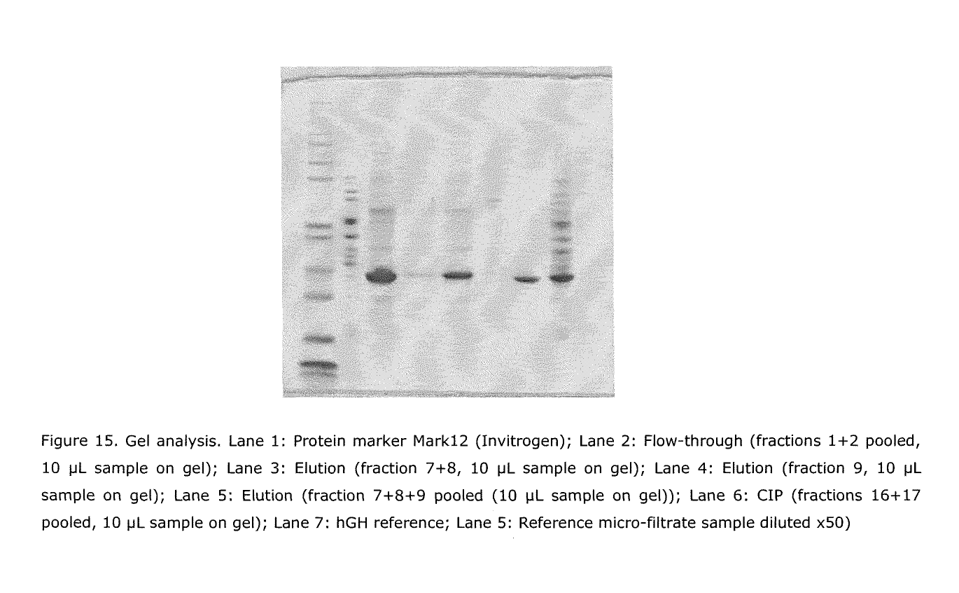

FIG. 16. HPLC chromatogram of pooled elution fractions 7, 8 and 9.

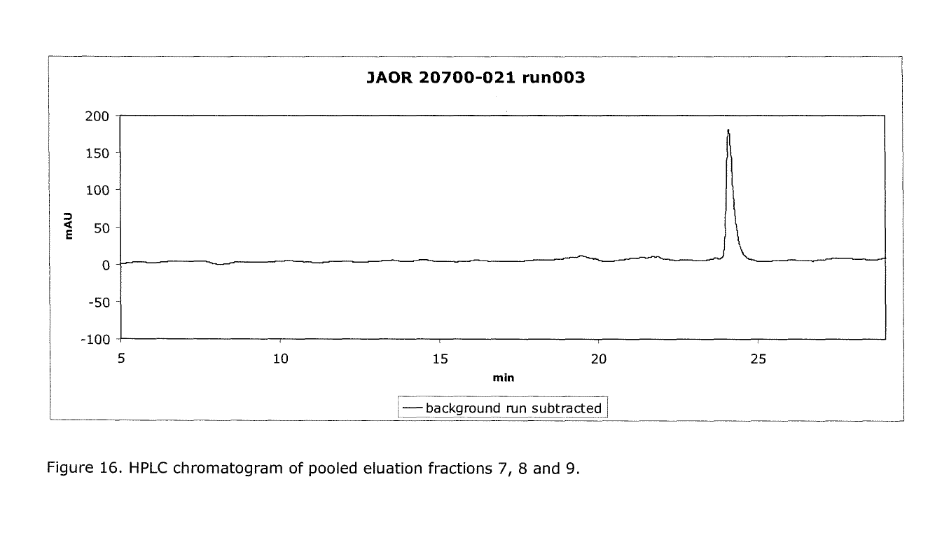

FIG. 17. Chromatogram from affinity purification of hyperglycosylated hGH using ligand L14 on Fractogel.

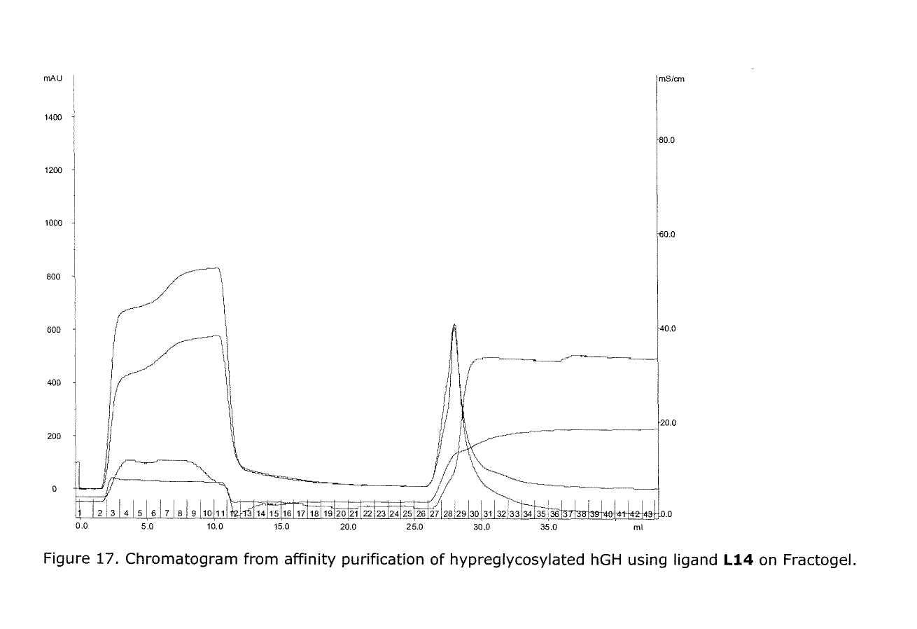

### DETAILED DESCRIPTION OF THE INVENTION

The present invention provides a process for the purification of a Growth Hormone polypeptide.

The current invention discloses a process for the purification of Growth Hormone (GH) polypeptide involving the use of affinity ligands and affinity resins wherein the ligands are specific binding partners of a Growth Hormone polypeptide and can therefore be used for the purification of said polypeptide.

Accordingly, one embodiment of present invention relates to a process for purification of growth hormone polypeptide said process comprising the steps of:

(a) contacting the growth hormone polypeptide with an affinity resin under conditions which facilitate binding of a portion of said growth hormone polypeptide to said affinity resin;

(b) optionally washing said affinity resin containing bound growth hormone polypeptide with a washing buffer; and

(c) eluting said affinity resin containing growth hormone polypeptide with an elution buffer, and collecting a purified Growth Hormone polypeptide as an eluate.

In one embodiment of present invention, the affinity resin is a solid phase material having covalently immobilized thereto ligands of the general formula (I),

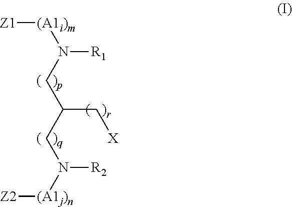

wherein

i=1, 2, . . . , m, and j=1, 2, . . . , n;

m and n are independently an integer in the range of 0-3, with the proviso that the sum n+m is in the range of 1-4;

p, q, and r are independently an integer in the range of 0-6;

A11, . . . , A1m and A21, . . . , A2n are independently selected from α-amino acid moieties, β-amino acid moieties, α-amino sulphonic acid moieties, and β-amino sulphonic acid moieties;

Z1 and Z2 are independently selected from hydrogen, C₁₋₆ alkyl, carboxylic acid moieties (Z—C(═O)—), and sulphonic acid moieties (Z—S(═O)₂—), wherein Z is selected from hydrogen, optionally substituted C₁₋₁₂-alkyl, optionally substituted C₃₋₁₂-cycloalkyl, optionally substituted C₁₋₁₂-alkenyl, optionally substituted C₁₋₁₂-alkynyl, optionally substituted aryl, optionally substituted heteroaryl and optionally substituted heterocyclyl;

R1 and R2 are independently selected from hydrogen and C₁₋₆-alkyl;

X is the group for attachment of the ligand to the solid phase material, either directly or via a linker, X being selected from carboxylic acid (—COOH), a carboxylic acid ester (—COOR), a carboxylic acid anhydride (—COOCOR), a carboxylic acid halide (—COHal), sulphonic acid (—S(═O)₂OH), a sulphonyl chloride (—S(═O)₂Cl), thiol (—SH), a disulphide (—S—S—R), hydroxy (—OH), aldehyde (C(═O)H), epoxide (—CH(O)CH₂), cyanide (—CN), halogen (-Hal), primary amine (—NH₂), secondary amine (—NHR), hydrazide (—NH═NH₂), and azide (—N₃), wherein R is selected from optionally substituted C₁₋₁₂-alkyl, and Hal is a halogen; and

the total molecular weight of said ligand (excluding “X” and any linker) being 200-2000 g/mol.

It has surprisingly been found that the scaffolds based on e.g. α,ω-diamino-carboxylic acids and similar types, such as α,β-diamino-propionic acid (p=1, q=r=0), α,γ-diamino-butyric acid, and α,δ-diamino-pentoic acid, in particular α,β-diamino-propionic acid (p=1, q=r=0), offers interesting classes of ligands which have excellent binding properties towards hGH polypeptides, and which further represents a specific binding to hGH polypeptides compared to other proteins present in, e.g., cell culture supernatants or plasma.

Additionally, and also supported by the above hypothesis, the ligand excluding “X” and any linker preferably has a molecular weight of more than 200 Da, such as more than 300 Da, for example more than 400 Da, such as more than 500 Da, for example more than 600 Da, such as more than 700 Da, for example a molecular weight of more than 800 Da. Independently thereof, the ligand preferably has a molecular weight of less than 5000 Da, such as less than 4000 Da, for example less than 3000 Da, such as less than 2500 Da, for example less than 2000 Da, such as less than 1500 Da, for example a molecular weight of less than 1000 Da.

In one embodiment of present invention, each of Z1-(A1i)ₘ-N(R1)- and Z2-(A2j)ₙ-N(R2)- represents an organic moiety of a molecular weight of 50-500 g/mol, wherein the total molecular weight of the ligand is 250-1500 g/mol, such as 300-1200 g/mol, e.g. 350-1000 g/mol.

In one embodiment of present invention, A11, . . . , A1m, and A21, . . . , A2n are independently selected from α-amino acid moieties and β-amino acid moieties, in particular from α-amino acid moieties.

In one embodiment of present invention, A11, . . . , A1m and A21, . . . , A2n are independently selected from the following amino acids in either of the L and D forms: Tyr, Phe, Arg, Trp, Ile, Pro, Thr, Lys, Gln, Asn, and Asp.

In embodiment of present invention, A11, . . . , A1m and A21, . . . , A2n are independently selected from D-Tyr, D-Phe, D-Arg, D-Trp, D-Ile, D-Pro, D-Thr, D-Lys, D-Gln, D-Asn, and D-Asp.

In embodiment of present invention, A11, . . . , A1m and A21, . . . , A2n are independently selected from L-Tyr, L-Phe, L-Arg, L-Trp, L-Ile, L-Pro, L-Thr, L-Lys, L-Gln, L-Asn, and L-Asp.

In one embodiment of present in invention, at least one of A11, . . . , A1m and A21, . . . , A2n is selected from L-Tyr, L-Phe, L-Arg, L-Trp, L-Ile, L-Pro, L-Thr, L-Lys, L-Gln, L-Asn, and L-Asp.

In one embodiment of present invention, A1₁, . . . , A1ₘ, A2₁, . . . , A2ₙ include at least one amino acid moiety selected from Arg, Phe, Ile, and Tyr, in particular at least one amino acid moiety selected from L-Arg, L-Phe, and L-Ile.

In one embodiment of present invention, Z1 and Z2 are independently selected from hydrogen, C₁₋₆ alkyl, carboxylic acid moieties and sulphonic acid moieties.

In one embodiment of present invention, Z1 and Z2 include at least one carboxylic acid moiety, or sulphonic acid moiety, in particular at least one carboxylic acid moiety.

In another embodiment of present invention, Z1 and Z2 are independently selected from hydrogen, C₁₋₆ alkyl, xanthene-9-yl-carbonyl, 5-methyl-2-phenyl-2H-1,2,3-triazole-4-yl-carbonyl, 3-amino-(phenylsulfonyl)-thiophen-2-yl-carbonyl, (+/−)-3-oxo-1-indanyl-carbonyl, 5,6,7,8-tetrahydroacridine-9-yl-carbonyl, and 2-methylimidazo[1,2-a]pyridine-3-yl-carbonyl.

Particularly preferred examples are those selected from the table given below (structures of corresponding carboxylic acid are provided for clarity):

| | |
|---|---|
| Xanthene-9-carbonyl, | 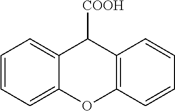 |
| 2-Amino nicotinyl, | 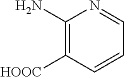 |
| 2-Quinaldincarbonyl, | 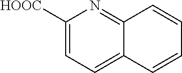 |
| 4,8-Dihydroxy-2-quinolinecarbonyl, | 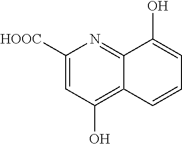 |
| 4-Quinolinecarbonyl, | 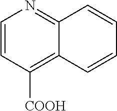 |
| 5-Methyl-2-nitrobenzoyl, | 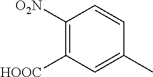 |
| 2-(Benzoimidazolylthio)acetyl, | 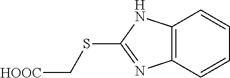 |
| 5-Methyl-2-phenyl-2H-1,2,3- triazole-4-carbonyl, | 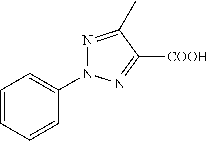 |
| 6-Hydroxy-2-naphthoyl, | 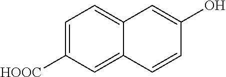 |
| 4,7-Dimethylpyrazolo[5,1-c] [1,2,4]triazine-3-carbonyl, | 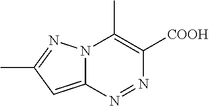 |
| 3-Amino-4-(phenylsulfonyl)- 2-thiophenecarbonyl, | 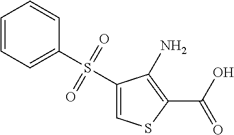 |
| (+/−)-3-Oxo-1-indancarbonyl, | 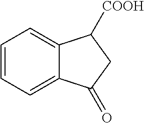 |
| 5,6,7,8-Tetrahydroacridine- 9-carbonyl, | 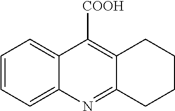 |
| 2-Methylimidazo[1,2-a]pyridine- 3-carbonyl, | 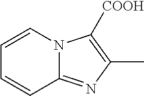 |
| 5-(4-Methyl-2-nitrophenyl)furoyl, | 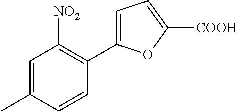 |
| 1-Cyclohexyl-4-oxo-1,4- dihydroquinoline-3-carbonyl, | 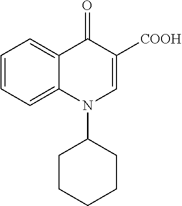 |
| Quinoxaline-6-carbonyl, and | 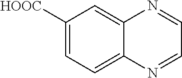 |
| 4-Methyl-2-phenylpyrimidine- 5-carbonyl. | 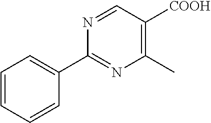 |

In one embodiment of present invention, Z1 is selected from the 1,2,3,4-tetrahydroacridine-9-yl-carbonyl, xanthen-9-yl-carbonyl, 2-methylimidazo[1,2-a]pyridine-3-yl-carbonyl, and 3-oxo-indan-1-yl-carbonyl.

In one embodiment of present invention, Z1 comprises a tricyclic heteroaromatic group, e.g. Z1 is 5,6,7,8-tetrahydroacridine-9-yl-carbonyl or xanthene-9-yl-carbonyl.

In one embodiment of the present invention, with respect to the number of amino acids/amino sulphonic acids, n is preferably 0-2, such as 0-1, in particular 0, and m is preferably 1-3, such as 2-3, in particular 2. The sum n+m is preferably 2-4, such as 2-3, in particular 2 or 3.

In one embodiment of present invention, when n is 0, Z2 is preferably selected from carboxylic acid moieties and sulphonic acid moieties. Also, when m is 0, Z1 is preferably selected from carboxylic acid moieties and sulphonic acid moieties.

In one embodiment of present invention, with respect to the number of carbon atoms in the scaffold, p is preferably 0-3, such as 0-2, such as 1-2, in particular 2, and q is preferably 0-3, such as 0-2, in particular 0 or 1. The sum p+q is preferably 1-7, such as 2-5, in particular 2 or 3. Independently thereof r is preferably 0-6, such as 0-4, such as 0-2, in particular 0 or 1, more particular 0.

In one embodiment of present invention, further variants with respect to the number of carbon atoms in the scaffold, (p,q) is (0,1), (0,2), (0,3), (0,4), (1,0), (2,0), (3,0), or (4,0).

In one embodiment of present invention, the r variant hereof is preferably 0.

In one embodiment of present, X is the reactive group used for attaching the ligand to the solid phase material, either directly or via a linker (see further below). In preferred embodiments the linker is attached to the scaffold via a segment —CON(R)— (in particular —NHCO—), where the carbonyl is a part of the scaffold representing “X”, because this will render it possible to prepare the ligand by standard methodologies known from solid phase peptide synthesis.

In one embodiment of present invention, X is typically selected from carboxylic acid (—COOH), a carboxylic acid ester (—COOR), a carboxylic acid anhydride (—COOCOR), a carboxylic acid halide (—COHal), sulphonic acid (—S(═O)₂OH), a sulphonyl chloride (—S(═O)₂Cl), thiol (—SH), a disulphide (—S—S—R), hydroxy (—OH), aldehyde (C(═O)H), epoxide (—CH(O)CH₂), cyanide (—CN), halogen (-Hal), primary amine (—NH₂), secondary amine (—NHR), hydrazide (—NH═NH₂), and azide (—N₃)0, wherein R is selected from optionally substituted C₁₋₁₂-alkyl, and Hal is a halogen.

In embodiment of present invention, X is COOH.

In embodiment of present invention the affinity resins comprising a solid phase material having a ligand of the general formula (I) attached optionally via a linker, wherein

A21, . . . , A2n are independently selected from Tyr, Phe, Arg, Trp, Ile, Pro, Thr, Lys, Gln, Asn and Asp, in particular from L-Tyr, L-Phe, L-Arg, L-Trp, L-Ile, L-Pro, L-Thr, L-Lys, L-Gln, L-Asn and L-Asp;

Z1 is selected from hydrogen, C₁₋₆ alkyl, xanthene-9-carbonyl, 2-amino nicotinyl, 2-quinaldincarbonyl, 4,8-dihydroxy-2-quinolinecarbonyl, 4-quinolinecarbonyl, 5-methyl-2-nitrobenzoyl, 2-(benzoimidazolylthio)acetyl, 5-methyl-2-phenyl-2H-1,2,3-triazole-4-carbonyl, 6-hydroxy-2-naphthoyl, 4,7-dimethylpyrazolo[5,1-c][1,2,4]triazine-3-carbonyl, 3-amino-4-(phenylsulfonyl)-2-thiophenecarbonyl, (+/−)-3-oxo-1-indancarbonyl, 5,6,7,8-tetrahydroacridine-9-carbonyl, 2-methylimidazo[1,2-a]pyridine-3-carbonyl, 5-(4-methyl-2-nitrophenyl)furoyl, 1-cyclohexyl-4-oxo-1,4-dihydroquinoline-3-carbonyl, quinoxaline-6-carbonyl, and 4-methyl-2-phenylpyrimidine-5-carbonyl;

Z2 is H;

R1 and R2 are independently selected from hydrogen and C₁₋₆-alkyl;

m=0, n=2, p=1, q=0, r=0;

are preferred.

In one embodiment of present invention, the affinity resins comprising a solid phase material having a ligand of the general formula (I) attached optionally via a linker, wherein

A21, . . . , A2n independently are selected from Tyr, Phe, Arg, Trp, Ile, Pro, Thr, Lys, Gln, Asn and Asp, in particular from L-Tyr, L-Phe, L-Arg, L-Trp, L-Ile, L-Pro, L-Thr, L-Lys, L-Gln, L-Asn and L-Asp;

Z1 is selected from hydrogen, C₁₋₆ alkyl, xanthene-9-yl-carbonyl, 5-methyl-2-phenyl-2H-1,2,3-triazole-4-yl-carbonyl, 3-amino-(phenylsulfonyl)-thiophen-2-yl-carbonyl, (+/−)-3-oxo-1-indanyl-carbonyl, 5,6,7,8-tetrahydroacridine-9-yl-carbonyl, and 2-methylimidazo[1,2-a]pyridine-3-yl-carbonyl;

Z2 is H;

R1 and R2 are independently selected from hydrogen and C₁₋₆-alkyl; m=0, n=2, p=1, q=0, r=0;

are preferred.

In one embodiment of present invention, the ligand has the general formula (II),

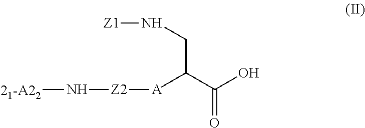

wherein Z1, Z2, A21, and A22 are as defined above for general formula (I), including the embodiments described for this general formula.

Particularly preferred are those ligands of the general formula (II), wherein

Z1 is Z—C(═O)—, wherein Z is selected from optionally substituted aryl, optionally substituted heteroaryl and optionally substituted heterocyclyl;

Z2 is selected from hydrogen and Z—C(═O)—, wherein Z is selected from optionally substituted C₁₋₁₂-alkyl, optionally substituted C₃₋₁₂-cycloalkyl, optionally substituted aryl, optionally substituted heteroaryl and optionally substituted heterocyclyl; and

each of A2₁ and A2₂ is independently selected from α-amino acids and β-amino acids.

In one embodiment of present invention, the ligand has the general formula (II), where A2₁ is preferably selected from arginine, phenylalanine, tyrosine, isoleucine, and lysine, and A2₂ is selected from arginine, phenylalanine, isoleucine, proline, tyrosine, and tryptophan.

One embodiment of present invention the ligand of general formula (II) has the preferred general formula (III),

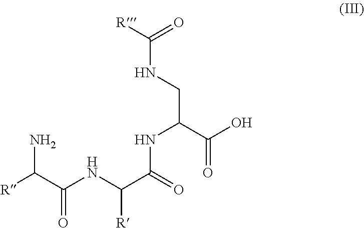

wherein R′ and R″ are independently selected from side chains of α-amino acids, and R″′ is selected from the group consisting of optionally substituted aryl, optionally substituted heteroaryl and optionally substituted heterocyclyl.

In one embodiment of present invention, the ligand has a structure selected from the following ligands Nos. (1)-(16):

| No. | Structure |
|---|---|
| 1 | 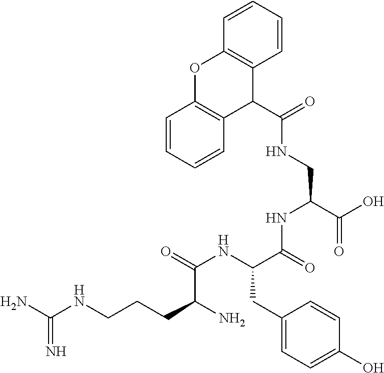 |
| 2 | 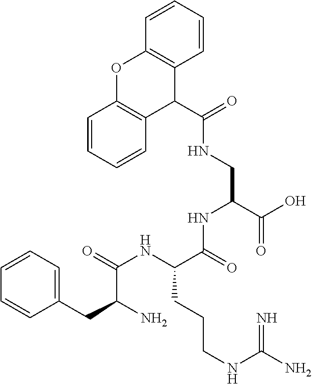 |
| 3 | 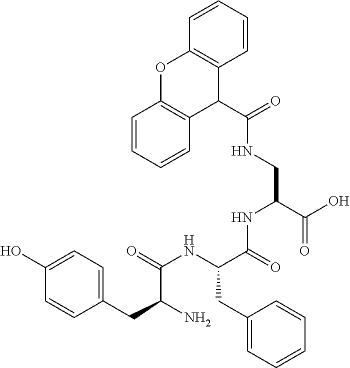 |
| 4 | 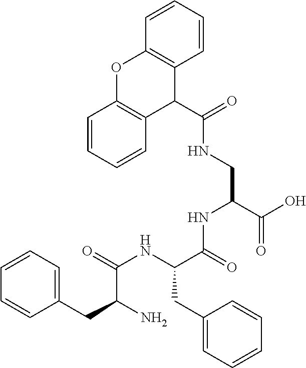 |
| 5 | 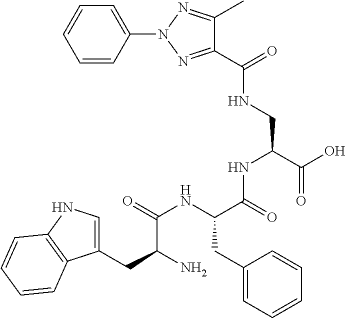 |
| 6 | 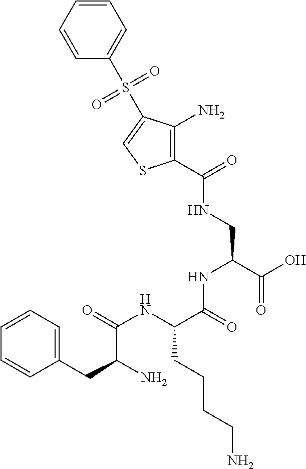 |
| 7 | 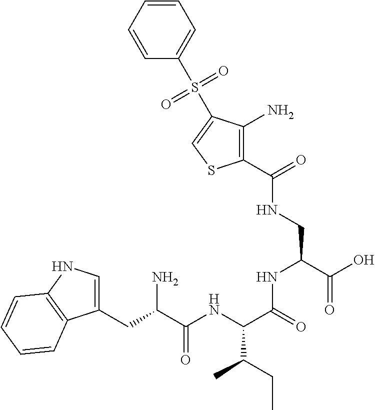 |
| 8 | 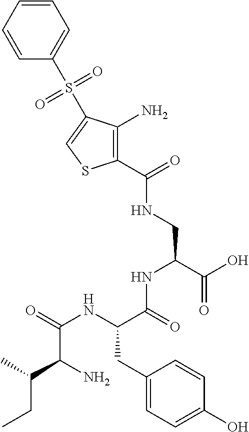 |
| 9 | 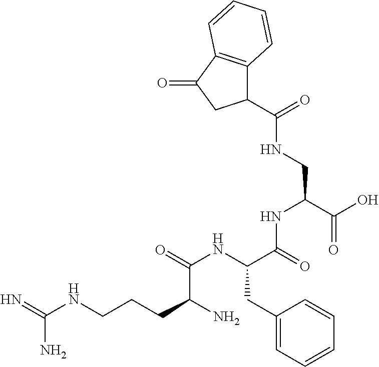 |
| 10 | 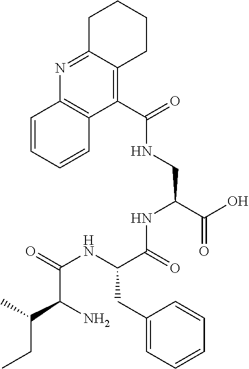 |
| 11 | 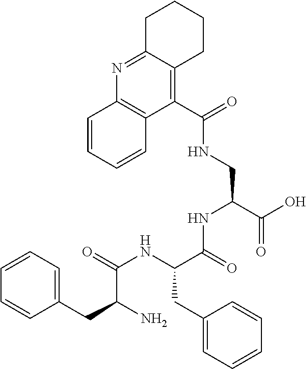 |
| 12 | 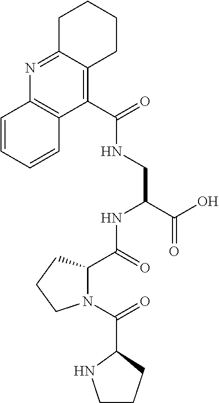 |
| 13 | 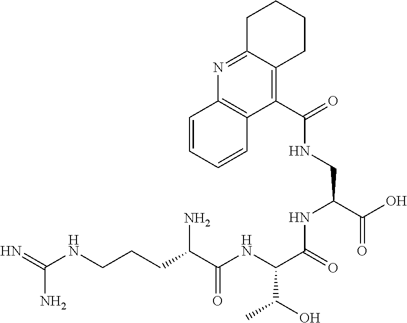 |
| 14 | 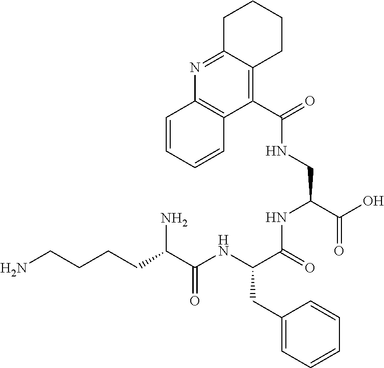 |
| 15 | 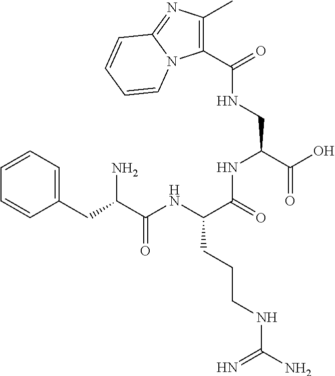 |
| 16 | 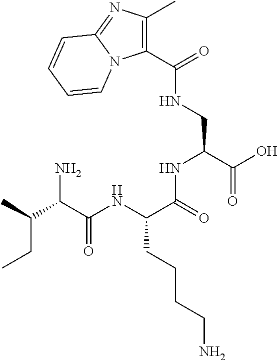 |

In a further preferred embodiment, the ligand has the general formula (I), wherein Z1 and Z2 are independently selected from hydrogen, C₁₋₆ alkyl, 5,6,7,8-tetrahydroacridine-9-carbonyl and 2-methylimidazo[1,2-a]pyridine-3-carbonyl.

The ligands described herein include enriched or resolved optical isomers at any or all asymmetric atoms as are apparent from the description or depiction herein. Both racemic and diasteromeric mixtures, as well as the individual optical isomers can be isolated or synthesized so as to be substantially free of their enantiomeric or diastereomeric counterparts, and these are all within the scope of the invention.

### DEFINITIONS

“α-amino acids moieties” refer to naturally occurring and synthetic amino acids (including the essential amino acids) wherein the amino group is covalently bonded to the α-carbon. When present in the ligands of the invention, the α-amino acid moieties are present as a —N(R)—X—C(═O)— fragment where X represent the α-carbon and any side chain(s).

“β-Amino acids moieties” refer to naturally occurring and synthetic amino acids wherein the amino group is covalently bonded to the β-carbon. When present in the ligands of the invention, the β-amino acid moieties are present as a —N(R)—X—C(═O)— fragment where X represent the α- and β-carbons and any side chain(s).

“α-amino sulphonic acid moieties” corresponds to “α-amino acids moieties” wherein the carbonyl group (—C(═O)—) has been replaced with a sulphonic group (—S(═O)₂—). When present in the ligands of the invention, the α-amino sulphonic acid moieties are present as a —N(R)—X—S(═O)₂— fragment where X represent the α-carbon and any side chain(s).

“β-amino sulphonic acid moieties” corresponds to “β-amino acids moieties” wherein the carbonyl group (—C(═O)—) has been replaced with a sulphonic group (—S(═O)₂—). When present in the ligands of the invention, the β-amino sulphonic acid moieties are present as a —N(R)—X—S(═O)₂— fragment where X represent the α- and β-carbons and any side chain(s).

The term “essential amino acid” refers to any one of the 20 genetically encoded L-α-amino acids and their stereoisomeric D-α-amino acids. Hence, the term “amino acid moieties” within the scope of the present invention is used in its broadest sense and is meant to include naturally-occurring L-amino acids thereof. The commonly used one- and three-letter abbreviations for naturally-occurring amino acids are used herein (Lehninger, Biochemistry, 2d ed., pp. 71-92, (Worth Publishers: New York, 1975). The term also includes D-amino acids (and residues thereof) as well as chemically-modified amino acids, such as amino acid analogues, including naturally-occurring amino acids that are not usually incorporated into proteins, such as norleucine, as well as chemically-synthesized compounds having properties known in the art to be characteristic of an amino acid.

Examples of amino acids that are generally capable of being incorporated into ligands according to the present invention as “amino acid moieties” are listed herein below:

Glycyl (GLY); aminopolycarboxylic acids, e.g., aspartic acid (ASP), p-hydroxyaspartic acid, glutamic acid (GLU), β-hydroxyglutamic acid, β-methylaspartic acid, β-methylglutamic acid, β,β-dimethylaspartic acid, γ-hydroxyglutamic acid, β,γ-dihydroxyglutamic acid, β-phenylglutamic acid, γ-methyleneglutamic acid, 3-aminoadipic acid, 2-aminopimelic acid, 2-aminosuberic acid and 2-aminosebacic acid residues; glutamine (GLN); asparagine (ASN); arginine (ARG), lysine (LYS), β-aminoalanine, γ-aminobutyrine, ornithine (ORN), citruline, homoarginine, homocitrulline, 5-hydroxy-2,6-diaminohexanoic acid, diaminobutyric acid; histidine (HIS); α,α′-diaminosuccinic acid, α,α′-diaminoglutaric acid, α,α′-diaminoadipic acid, α,α′-diaminopimelic acid, α,α′-diamino-β-hydroxypimelic acid, α,α′-diaminosuberic acid, α,α′-diaminoazelaic acid, and α,α′-diaminosebacic acid residues; proline (PRO), 4- or 3-hydroxy-2-pyrrolidine-carboxylic acid, γ-methylproline, pipecolic acid, 5-hydroxypipecolic acid, —N[CH₂]₂CO—, azetidine-2-carboxylic acid; alanine (ALA), valine (VAL), leucine (LEU), allylglycine, butyrine, norvaline, norleucine (NLE), heptyline, α-methylserine, α-amino-α-methyl-γ-hydroxyvaleric acid, α-amino-α-methyl-6-hydroxyvaleric acid, α-amino-α-methyl-ε-hydroxycaproic acid, isovaline, α-methylglutamic acid, α-aminoisobutyric acid, α-aminodiethylacetic acid, α-aminodiisopropylacetic acid, α-aminodi-n-propylacetic acid, α-aminodiisobutylacetic acid, α-aminodi-n-butylacetic acid, α-aminoethylisopropylacetic acid, α-amino-n-propylacetic acid, α-aminodiisoamyacetic acid, α-methylaspartic acid, α-methylglutamic acid, 1-aminocyclopropane-1-carboxylic acid; isoleucine (ILE), alloisoleucine, tert-leucine, β-methyltryptophan; α-amino-α-ethyl-β-phenylpropionic acid; β-phenylserinyl; serine (SER), β-hydroxyleucine, β-hydroxynorleucine, β-hydroxynorvaline, α-amino-α-hydroxystearic acid; homoserine, γ-hydroxynorvaline, δ-hydroxynorvaline, ε-hydroxynorleucine; canavinyl, canalinyl; γ-hydroxyornithinyl; 2-hexosaminic acid, D-glucosaminic acid, D-galactosaminic acid; α-amino-β-thiols, penicillamine, β-thiolnorvaline, β-thiolbutyrine; cysteine (CYS); homocystine; β-phenylmethionine; methionine (MET); S-allyl-(L)-cysteine sulfoxide; 2-thiolhistidine; cystathionine;; phenylalanine (PHE), tryptophan (TRP), α-aminophenylacetic acid, α-aminocyclohexylacetic acid, α-amino-β-cyclohexylpropionic acid; aryl-, C₁₋₆-alkyl-, hydroxyl-, halogen-, guanidine-, oxyalkylether-, nitro-, sulphur- or halo-substituted phenyl (e.g., tyrosine (TYR), methyltyrosine and o-chloro-, p-chloro-, 3,4-dichloro, o-, m- or p-methyl-, 2,4,6-trimethyl-, 2-ethoxy-5-nitro, 2-hydroxy-5-nitro and p-nitro-phenylalanine); furyl-, thienyl-, pyridyl-, pyrimidinyl-, purine or naphthylalanines; kynurenine, 3-hydroxykynurenine, 2-hydroxytryptophan, 4-carboxytryptophan; sarcosine (N-methylglycine; SAR), N-benzylglycine, N-methylalanine, N-benzylalanine, N-methylphenylalanine, N-benzylphenylalanine, N-methylvaline and N-benzylvaline; threonine (THR), allothreonine, phosphoserine, phosphothreonine.

The term “side chains of α-amino acids” refers to the groups constituting the side chains of the amino acids disclosed hereinabove. Typically, such side chains are selected from hydrogen (representing glycine), methyl (alanine), 2-propyl (valine), 2-methyl-1-propyl (leucine), 2-butyl (isoleucine), methylthioethyl (methionine), benzyl (phenylalanine), 3-indolylmethyl (tryptophan), hydroxymethyl (serine), 1-hydroxyethyl (threonine), mercaptomethyl (cysteine), 4-hydroxybenzyl (tyrosine), aminocarbonylmethyl (asparagine), 2-aminocarbonylethyl (glutamine), carboxymethyl (aspartic acid), 2-carboxyethyl (glutamic acid), 4-amino-1-butyl (lysine), 3-guanidino-1-propyl (arginine), and 4-imidazolylmethyl (histidine), or the side chain together with the intervening carbon and neighboring nitrogen atom form a pyrrolidine ring (proline).

“α-amino sulphonic acid moieties” refers to “α-Amino acids moieties” wherein the carbonyl oxygen (═O) has been replaced with sulphur (═S). “β-amino sulphonic acid moieties” refers to “β-Amino acids moieties” wherein the carbonyl oxygen (═O) has been replaced with sulphur (═S).

In the present context, the terms “C₁₋₁₂-alkyl” and “C₁₋₆-alkyl” are intended to mean a linear, cyclic or branched hydrocarbon group having 1 to 12 carbon atoms and 1 to 6 carbon atoms, respectively, such as methyl, ethyl, propyl, iso-propyl, pentyl, cyclopentyl, hexyl, cyclohexyl. The term “C₁₋₄-alkyl” is intended to cover linear, cyclic or branched hydrocarbon groups having 1 to 4 carbon atoms, e.g. methyl, ethyl, propyl, iso-propyl, cyclopropyl, butyl, iso-butyl, tert-butyl, cyclobutyl.

Although the term “C₃₋₁₂-cycloalkyl” is encompassed by the term “C₁₋₁₂-alkyl”, it refers specifically to the mono- and bicyclic counterparts, including alkyl groups having exo-cyclic atoms, e.g. cyclohexyl-methyl.

Similarly, the terms “C₂₋₁₂-alkenyl” and “C₂₋₆-alkenyl” are intended to cover linear, cyclic or branched hydrocarbon groups having 2 to 12 carbon atoms and 2 to 6 carbon atoms, respectively, and comprising (at least) one unsaturated bond. Examples of alkenyl groups are vinyl, allyl, butenyl, pentenyl, hexenyl, heptenyl, octenyl, heptadecaenyl. Preferred examples of alkenyl are vinyl, allyl, butenyl, especially allyl.

Although the term “C₃₋₁₂-cycloalkenyl” is encompassed by the term “C₂₋₁₂-alkenyl”, it refers specifically to the mono- and bicyclic counterparts, including alkenyl groups having exo-cyclic atoms, e.g. cyclohexenyl-methyl.

Similarly, the terms “C₂₋₁₂-alkynyl” and “C₂₋₆-alkynyl” are intended to cover linear, cyclic or branched hydrocarbon groups having 2 to 12 carbon atoms and 2 to 6 carbon atoms, respectively, and comprising (at least) one triple bond.

The term “C₁₋₆-alkoxy” is intended to mean “C₁₋₆-alkyl-O”.

In the present context, i.e. in connection with the terms “alkyl”, “alkoxy”, “alkenyl”, “alkynyl”, and the like, the term “optionally substituted” is intended to mean that the group in question may be substituted one or several times, preferably 1-3 times, with group(s) selected from hydroxy (which when bound to an unsaturated carbon atom may be present in the tautomeric keto form), C₁₋₆-alkoxy (i.e. C₁₋₆-alkyl-oxy), C₂₋₆-alkenyloxy, carboxy, oxo (forming a keto or aldehyde functionality), C₁₋₆-alkoxycarbonyl, C₁₋₆-alkylcarbonyl, formyl, aryl, aryloxy, arylamino, arylcarbonyl, aryloxycarbonyl, arylcarbonyloxy, arylaminocarbonyl, arylcarbonylamino, heteroaryl, heteroaryloxy, heteroarylamino, heteroarylcarbonyl, heteroaryloxycarbonyl, heteroarylcarbonyloxy, heteroarylaminocarbonyl, heteroarylcarbonylamino, heterocyclyl, heterocyclyloxy, heterocyclylamino, heterocyclylcarbonyl, heterocyclyloxycarbonyl, heterocyclylcarbonyloxy, heterocyclylaminocarbonyl, heterocyclylcarbonylamino, amino, mono- and di(C₁₋₆-alkyl)amino, —N(C₁₋₄-alkyl)₃⁺, carbamoyl, mono- and di(C₁₋₆-alkyl)aminocarbonyl, C₁₋₆-alkylcarbonylamino, cyano, guanidino, carbamido, C₁₋₆-alkyl-sulphonyl-amino, aryl-sulphonyl-amino, heteroaryl-sulphonyl-amino, C₁₋₆-alkanoyloxy, C₁₋₆-alkyl-sulphonyl, C₁₋₆-alkyl-sulphinyl, C₁₋₆-alkylsulphonyloxy, nitro, C₁₋₆-alkylthio, and halogen, where any aryl, heteroaryl and heterocyclyl may be substituted as specifically described below for aryl, heteroaryl and heterocyclyl, and any alkyl, alkoxy, and the like, representing substituents may be substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s).

Typically, the substituents are selected from hydroxy (which when bound to an unsaturated carbon atom may be present in the tautomeric keto form), C₁₋₆-alkoxy (i.e. C₁₋₆-alkyl-oxy), C₂₋₆-alkenyloxy, carboxy, oxo (forming a keto or aldehyde functionality), C₁₋₆-alkylcarbonyl, formyl, aryl, aryloxy, arylamino, arylcarbonyl, heteroaryl, heteroaryloxy, heteroarylamino, heteroarylcarbonyl, heterocyclyl, heterocyclyloxy, heterocyclylamino, heterocyclylcarbonyl, amino, mono- and di(C₁₋₆-alkyl)amino; carbamoyl, mono- and di(C₁₋₆-alkyl)amino-carbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, guanidino, carbamido, C₁₋₆-alkyl-sulphonyl-amino, C₁₋₆-alkyl-sulphonyl, C₁₋₆-alkyl-sulphinyl, C₁₋₆-alkylthio, halogen, where any aryl, heteroaryl and heterocyclyl may be substituted as specifically described below for aryl, heteroaryl and heterocyclyl.

In some embodiments, substituents are selected from hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen.

The term “halogen” includes fluoro, chloro, bromo, and iodo.

In the present context, the term “aryl” is intended to mean a fully or partially aromatic carbocyclic ring or ring system, such as phenyl, naphthyl, 1,2,3,4-tetrahydronaphthyl, biphenyl, anthracyl, phenanthracyl, pyrenyl, benzopyrenyl, fluorenyl and xanthenyl, among which phenyl is a preferred example.

The term “heteroaryl” is intended to mean a fully or partially aromatic carbocyclic ring or ring system where one or more of the carbon atoms have been replaced with heteroatoms, e.g. nitrogen (═N— or —NH—), sulphur, and/or oxygen atoms. Examples of such heteroaryl groups are oxazolyl, isoxazolyl, thiazolyl, isothiazolyl, pyrrolyl, imidazolyl, pyrazolyl, pyridinyl, pyrimidinyl, pyrazinyl, pyridazinyl, triazinyl, coumaryl, furanyl, thienyl, quinolyl, benzo-thiazolyl, benzotriazolyl, benzodiazolyl, benzooxozolyl, phthalazinyl, phthalanyl, triazolyl, tetrazolyl, isoquinolyl, acridinyl, carbazolyl, dibenzazepinyl, indolyl, benzopyrazolyl, phenoxazonyl. Particularly interesting heteroaryl groups are benzimidazolyl, oxazolyl, isoxazolyl, thiazolyl, isothiazolyl, pyrrolyl, imidazolyl, pyrazolyl, pyridinyl, pyrimidinyl, pyrazinyl, pyridazinyl, furyl, thienyl, quinolyl, triazolyl, tetrazolyl, isoquinolyl, indolyl in particular benzimidazolyl, pyrrolyl, imidazolyl, pyridinyl, pyrimidinyl, furyl, thienyl, quinolyl, tetrazolyl, and isoquinolyl.

The term “heterocyclyl” is intended to mean a non-aromatic carbocyclic ring or ring system where one or more of the carbon atoms have been replaced with heteroatoms, e.g. nitrogen (═N— or —NH—), sulphur, and/or oxygen atoms. Examples of such heterocyclyl groups (named according to the rings) are imidazolidine, piperazine, hexahydropyridazine, hexahydropyrimidine, diazepane, diazocane, pyrrolidine, piperidine, azepane, azocane, aziridine, azirine, azetidine, pyrroline, tropane, oxazinane (morpholine), azepine, dihydroazepine, tetrahydroazepine, and hexahydroazepine, oxazolane, oxazepane, oxazocane, thiazolane, thiazinane, thiazepane, thiazocane, oxazetane, diazetane, thiazetane, tetrahydrofuran, tetrahydropyran, oxepane, tetrahydrothiophene, tetrahydrothiopyrane, thiepane, dithiane, dithiepane, dioxane, dioxepane, oxathiane, oxathiepane. The most interesting examples are tetrahydrofuran, imidazolidine, piperazine, hexahydropyridazine, hexahydropyrimidine, diazepane, diazocane, pyrrolidine, piperidine, azepane, azocane, azetidine, tropane, oxazinane (morpholine), oxazolane, oxazepane, thiazolane, thiazinane, and thiazepane, in particular tetrahydrofuran, imidazolidine, piperazine, hexahydropyridazine, hexahydropyrimidine, diazepane, pyrrolidine, piperidine, azepane, oxazinane (morpholine), and thiazinane.

In the present context, i.e. in connection with the terms “aryl”, “heteroaryl”, “heterocyclyl” and the like (e.g. “aryloxy”, “heterarylcarbonyl”, etc.), the term “optionally substituted” is intended to mean that the group in question may be substituted one or several times, preferably 1-5 times, in particular 1-3 times, with group(s) selected from hydroxy (which when present in an enol system may be represented in the tautomeric keto form), C₁₋₆-alkyl, C₁₋₆-alkoxy, C₂₋₆-alkenyloxy, oxo (which may be represented in the tautomeric enol form), oxide (only relevant as the N-oxide), carboxy, C₁₋₆-alkoxycarbonyl, C₁₋₅-alkylcarbonyl, formyl, aryl, aryloxy, arylamino, aryloxycarbonyl, arylcarbonyl, heteroaryl, heteroarylamino, amino, mono- and di(C₁₋₆-alkyl)amino; carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, cyano, guanidino, carbamido, C₁₋₆-alkanoyloxy, C₁₋₆-alkyl-sulphonyl-amino, aryl-sulphonyl-amino, heteroaryl-sulphonyl-amino, C₁₋₆-alkyl-sulphonyl, C₁₋₆-alkyl-sulphinyl, C₁₋₆-alkylsulphonyloxy, nitro, sulphanyl, amino, amino-sulfonyl, mono- and di(C₁₋₆-alkyl)amino-sulfonyl, dihalogen-C₁₋₄-alkyl, trihalogen-C₁₋₄-alkyl, halogen, where aryl and heteroaryl representing substituents may be substituted 1-3 times with C₁₋₄-alkyl, C₁₋₆-alkoxy, nitro, cyano, amino or halogen, and any alkyl, alkoxy, and the like, representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino.

Typically, the substituents are selected from hydroxy, C₁₋₆-alkoxy, oxo (which may be represented in the tautomeric enol form), carboxy, C₁₋₆-alkylcarbonyl, formyl, amino, mono- and di(C₁₋₆-alkyl)amino; carbamoyl, mono- and di(C₁₋₆-alkyl)aminocarbonyl, amino-C₁₋₆-alkyl-aminocarbonyl, C₁₋₆-alkylcarbonylamino, guanidino, carbamido, C₁₋₆-alkyl-sulphonyl-amino, aryl-sulphonyl-amino, heteroaryl-sulphonyl-amino, C₁₋₆-alkyl-sulphonyl, C₁₋₆-alkyl-sulphinyl, C₁₋₆-alkylsulphonyloxy, sulphanyl, amino, amino-sulfonyl, mono- and di(C₁₋₆-alkyl)amino-sulfonyl or halogen, where any alkyl, alkoxy and the like, representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino. In some embodiments, the substituents are selected from C₁₋₆-alkyl, C₁₋₆-alkoxy, amino, mono- and di(C₁₋₆-alkyl)amino, sulphanyl, carboxy or halogen, where any alkyl, alkoxy and the like, representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino.

Carboxylic acid moieties refer to moieties which are included as Z1 and Z2 as Q-C(═O)—, where Q is selected from optionally substituted C₁₋₁₂-alkyl, optionally substituted C₂₋₁₂-alkenyl, optionally substituted C₂₋₁₂-alkynyl, optionally substituted aryl, optionally substituted heteroaryl, and optionally substituted heterocyclyl.

Sulphonic acid moieties refer to moieties which are included as Z1 and Z2 as Q-S(═O)₂—, where Q is selected from optionally substituted C₁₋₁₂-alkyl, optionally substituted C₂₋₁₂-alkenyl, optionally substituted C₂₋₁₂-alkynyl, optionally substituted aryl, optionally substituted heteroaryl, and optionally substituted heterocyclyl.

When used herein, the expression “organic moiety” (and “organic moieties”) is intended to mean a molecular fragment comprising one or more carbon atoms and one or more hydrogen (H), oxygen (O), nitrogen (N), sulphur (S), bromine (Br), chlorine (Cl), fluorine (F), or phosphor (P) atoms covalently bonded.

In certain aspects of the invention, each of the organic moieties Z1-(A1i)ₘ-N(R1)- and Z2-(A2i)ₙ-N(R2)- typically have the general formula CₐH_{b}O_{c}N_{d}SₑBr_{f}Cl_{g}Fₕ,Pₗ wherein 0≦a≦15, 0≦b≦2x+1, 0≦c≦x, 0≦d≦x, 0≦e≦x, 0≦f≦3x, 0≦g≦3x, 0≦h≦3x, 0≦i≦x and 50≦12a+b+16c+14d+32e+80f+35g+19h+31i≦5500.

### Solution or Suspension

The expression “solution or suspension” herein is intended to mean a solid mass or/and liquid mass, comprising the Growth Hormone polypeptide, in particular a human Growth Hormone polypeptide. The expression “solution or suspension” is in particular meant to refer to a “large” volume or mass, i.e. referring to volumes and masses known from large-scale and industrial-scale processes.

The “suspension or solution of the Growth Hormone polypeptide typically originates from for e.g. a cell culture, a microbial process, a cloned animal (e.g. cows, pigs, sheep, goats, and fish) or insect, or the like, in particular from a cell culture or an industrial-scale production process. Alternatively, the suspension or solution of the Growth Hormone polypeptide may be derived from blood plasma, or the like.

The suspension or solution of the Growth Hormone polypeptide is typically obtained after lysing of cells in a particular cell culture or directly from cell culture fluid. The suspension or solution containing the Growth Hormone polypeptide can be subsequently adjusted by changing pH, ionic strength, or by chelation of divalent metal ions, etc., if desirable or beneficial.

In one embodiment of present invention, the suspension or solution containing the Growth Hormone polypeptide is obtained directly from a preceding purification step, or from a preceding purification step with subsequent adjustment of pH, ionic strength, chelation of divalent metal ions, etc., if desirable or beneficial.

### Ligands

The affinity resin is a solid phase material (see below) having covalently immobilized thereto ligands that have a high specificity towards the Growth Hormone polypeptide as described in the context of present invention.

When used herein, the term “ligand” means a molecule which can bind a target compound which, in the present context, can be a Growth Hormone polypeptide. Preferably, the ligands should bind to the Growth Hormone polypeptide in question at least in a substantially specific manner (“specific binding”). The expression “one or more ligands” refers to the fact that the solid phase material may have more than one type of ligand immobilized thereto. This being said, immobilization of a single type of ligands (“a ligand”) will typically involve the immobilization of a plurality/multitude of species of identical ligands.

In the present context, “specific binding” refers to the property of a ligand to bind to a Growth Hormone polypeptide (i.e. the binding partner), preferentially such that the relative mass of bound Growth Hormone polypeptide, is at least two-fold, such as 50-fold, for example 100-fold, such as 1000-fold, or more, greater than the relative mass of other bound species than the Growth Hormone polypeptide. By relative mass of bound compound is meant the relative mass of bound specific binder=(mass specific bound/total compound bound)/(mass of bound non-specific/total compound bound).

The affinity ligands were designed in silico by a virtual combinatorial screening procedure to bind to the hGHbp-binding epitope of hGH polypeptide known as Site 1. The ligands are particularly designed to interact with the residues Glu56, Ile58, Thr60, Pro61, Ser62, Asn63, Arg64, Thr67, Gln68, Gln69, Lys168, Asp171, Lys172, Thr175, Phe176, and Ile179 in the Site 1 epitope on hGH. hGH interacts with its receptor (hGHbp) through binding first at the high affinity Site 1 of hGH polypeptide and subsequently with a second molecule of hGHbp at the lower affinity Site 2 to form a 2:1 receptor-ligand complex (Walsh et al., Proc. Natl. Acad. Sci. 2004, 101, 17078-17083). The binding affinity of hGH polypeptide towards hGHbp at Site 1 is predominately facilitated by two tryptophan-residues on hGHbp, namely Trp104 and Trp169, both located in a central hydrophobic patch within the larger Site 1. These residues account for the majority of the binding affinity to hGH, with hGHbp mutants W104A and W169A showing reduced affinity relative to the wild-type by a factor of more than 2500 respectively (Clackson et al., J. Mol. Biol. 1998, 277, 1111-1128). The ligands described herein have been so designed as to mimic these favourable interactions by computational docking of a large number of combinatorially constructed ligands into the high-affinity patch of Site 1 which interacts with Trp104 and Trp169 of hGHbp, namely the residues Glu56, Ile58, Thr60, Pro61, Ser62, Asn63, Arg64, Thr67, Gln68, Gln69, Lys168, Asp171, Lys172, Thr175, Phe176, and Ile179 in the Site 1 epitope on hGH.

### Solid Phase Material

As mentioned above, the affinity resin is a solid phase material substituted having immobilized thereto one or more synthetic ligands. The solid phase material (sometimes referred to as “a matrix” or “a polymer matrix”) may in principle be selected from a broad range of the materials conventionally use for chromatographic purposes and for peptides synthesis. Examples of such materials are described below.

The ligand is covalently immobilized to a solid phase material such as a porous, inorganic matrix or a polymer matrix, optionally in cross-linked and/or beaded form or in a monolithic porous entity. Preferably, the pores of the polymer matrix are sufficiently wide for the target protein to diffuse through said pores and interact with the ligand on the inner surface of the pores. For a GH polypeptide with molar mass approx. 22 kDa an average pore diameter of 20-150 nm is preferred, such as approx. 75 nm.

The beaded and optionally cross-linked polymer matrix in one embodiment comprises a plurality of hydrophilic moieties. The hydrophilic moieties can be polymer chains which, when cross-linked, form the cross-linked polymer matrix. Examples include e.g. polyethylene glycol moieties, polyamine moieties, polyvinylamine moieties, and polyol moieties.

In one embodiment of present invention, the core and/or the surface of a beaded polymer matrix comprises a polymeric material selected from the group consisting of polyvinyls, polyacrylates, polyacrylamides, polystyrenes, polyesters and polyamides.

The beaded polymer matrix can also be selected from the group consisting of PS, POEPS, POEPOP, SPOCC, PEGA, CLEAR, Expansin, Polyamide, Jandagel, PS-BDODMA, PS-HDODA, PS-TTEGDA, PS-TEGDA, GDMA-PMMA, PS-TRPGDA, ArgoGel, Argopore resins, ULTRAMINE, crosslinked LUPAMINE, high capacity PEGA, Silica, Fractogel, Sephadex, Sepharose, Glass beads, crosslinked polyacrylates, and derivatives of the aforementioned; in particular, the polymer matrix is selected from the group consisting of SPOCC, PEGA, HYDRA, POEPOP, PEG-polyacrylate copolymers, polyether-polyamine copolymers, and cross-linked polyethylene di-amines.

Apart from the above-mentioned examples, any material capable of forming a polymer matrix can in principle be used in the production of beads of the invention. Preferably, the core material of a bead is polymeric. In some embodiments, the core comprises or consists of hydrophilic polymeric material. In other embodiments, the core comprises or consists of hydrophobic polymeric material. In some embodiments, the surface of the beads comprises or consists of the same material as the core.

Resins useful for large-scale applications may be one of the above mentioned or other resins such as Sephadex™, Sepharose™, Fractogel™, CIMGEL™, Toyopearl, HEMA™, crosslinked agarose, crosslinked cellulose, other crosslinked-carbohydrate-based resins and macroporous polystyrene or polyacrylate. The matrix may also be of a mainly inorganic nature, such as macroporous glass or clay minerals, or combinations of resins and inorganics, such as Ceramic HyperD™ or silica gel.

Polymer beads according to the invention can be prepared from a variety of polymerisable monomers, including styrenes, acrylates and unsaturated chlorides, esters, acetates, amides and alcohols, including, but not limited to, polystyrene (including high density polystyrene latexes such as brominated polystyrene), polymethylmethacrylate and other polyacrylic acids, polyacrylonitrile, polyacrylamide, polyacrolein, polydimethylsiloxane, polybutadiene, polyisoprene, polyurethane, polyvinylacetate, polyvinylchloride, polyvinylpyridine, polyvinylbenzylchloride, polyvinyltoluene, polyvinylidenechloride and polydivinylbenzene. In other embodiments, the beads are prepared from styrene monomers or PEG based macro-monomers. The polymer is in preferred embodiments selected from the group consisting of polyethers, polyvinyls, polyacrylates, polymethacrylates, polyacylamides, polyurethanes, polyacrylamides, polystyrenes, polycarbonates, polyesters, polyamides, and combinations thereof. Highly preferred surface and core moieties include cross-linked PEG moieties, polyamine moieties, polyvinylamine moieties, and polyol moieties.

A preferred hydrophobic polymer to be used for production of beads of the composition of the invention is PS-DVB (polystyrene divinylbenzene). PS-DVB has been widely used for solid-phase peptide synthesis (SPPS), and has more recently demonstrated utility for the polymer-supported preparation of particular organic molecules (Adams et al. (1998) J. Org. Chem. 63:3706-3716). When prepared properly (Grøtli et al. (2000) J. Combi. Chem. 2:108-119), PS-DVB solid phase materials display excellent properties for chemical synthesis such as high loading, reasonable swelling in organic solvents and physical stability.

### Linkers

The above-mentioned ligand is covalently immobilized to a solid phase material, possibly through a linker. In preferred embodiments, the ligand is covalently attached to a linker, which is covalently attached to the polymer matrix. General techniques for linking of affinity ligands to solid phase materials can be found in Hermanson, Krishna Mallia and Smith, Immobilized Affinity Ligand Techniques”, Academic Press, 1992.

It should be understood that the linker should provide a suitable mobility of the ligand, but should not as such participate in the binding of the ligand to the antibody of interest. In fact, the binding of the immobilised ligand should be similar to the binding of the non-immobilised ligand.

Linkers are used for linking the ligand to a solid phase material such as e.g. a polymer matrix or an inorganic support. Preferably, the linker forms a strong and durable bond between the ligand and the solid phase material. This is particularly important, when the solid phase material of the present invention is to be used for repeated purification of GH polypeptides.

However, in one embodiment of the present invention, linkers can be selectively cleavable. This can be useful when the solid phase material is to be used for analytical purposes.

Amino acids and polypeptides are examples of typical linkers. Other possible linkers include carbohydrates and nucleic acids.

In one embodiment of present invention, the linker residue L attached to the polymer matrix is cleavable by acids, bases, temperature, light, or by contact with a chemical reagent. In particular, the linker attached to the polymer matrix can be (3-formylindol-1-yl)acetic acid, 2,4-dimethoxy-4′-hydroxy-benzophenone, HMPA, HMPB, HMPPA, Rink acid, Rink amide, Knorr linker, PAL linker, DCHD linker, Wang linker and Trityl linker.

The ligand can be associated with the solid phase material through a linker having a length of preferably less than 50 Å, such as a length of from 3 to 30 Å, for example a length of from 3 to 20 Å, such as a length of from 3 to 10 Å.

Preferably the linker is attached to the ligand via a carboxylic acid group, or an amino group, in particular via a carboxylic acid group.

The linker may also comprise a plurality of covalently linked subunits, e.g. such that the subunits are selected from identical and non-identical linker subunits. In one variant, the linker is flexible and comprises from 3 to preferably less than 50 identical or non-identical, covalently linked subunits.

In one embodiment of the present invention, the linker L is selected from the group consisting of glycine, alanine, 3-aminopropionic acid, 4-aminobutanoic acid, and HMBA.

In one embodiment of present invention, the linker can be selected from alkanes, such as linear alkanes, such as linear alkanes with 2-12 carbon atoms, monodisperse polyethyleneglycol (PEG), such as PEG with 2-20 repeat units, and peptides, such as peptides comprising 1-20 linked amino acids.

The linker can also be selected from the group consisting of polydispersed polyethylene glycol; monodispersed polyethylene glycol, such as triethylene glycol, tetraethylene glycol, pentaethylene glycol, hexaethylene glycol, heptaethylene glycol; an amino acid; a dipeptide; a tripeptide; a tetrapeptide; a pentapeptide; a hexapeptide; a heptapeptide; octapeptide; a nonapeptide; a decapeptide, a polyalanine; a polyglycine, including any combination thereof.

The solid phase material is most often presented in the form of beads, e.g. a particulate material having an average diameter of in the range of 0.1-1000 μm, or in the form of sticks, membranes, pellets, monoliths, etc.

### Peptide

The term “peptide” is intended to indicate a sequence of two or more amino acids joined by peptide bonds, wherein the individual amino acids may be naturally occurring or synthetic. The term encompasses the terms polypeptides and proteins, which may consists of two or more polypeptides held together by covalent interactions, such as for instance cysteine bridges, or non-covalent interactions. It is to be understood that the term is also intended to include peptides, which have been derivatized, for instance by the attachment of lipophilic groups, PEG or prosthetic groups. The term peptide includes any suitable peptide and may be used synonymously with the terms polypeptide and protein, unless otherwise stated or contradicted by context; provided that the reader recognize that each type of respective amino acid polymer-containing molecule may be associated with significant differences and thereby form individual embodiments of the present invention (for example, a peptide such as an antibody, which is composed of multiple polypeptide chains, is significantly different from, for example, a single chain antibody, a peptide immunoadhesin, or single chain immunogenic peptide). Therefore, the term peptide herein should generally be understood as referring to any suitable peptide of any suitable size and composition (with respect to the number of amino acids and number of associated chains in a protein molecule). Moreover, peptides described herein may comprise non-naturally occurring and/or non-L amino acid residues, unless otherwise stated or contradicted by context.

The term peptide, unless otherwise stated or contradicted by context, (and if discussed as individual embodiments of the term(s) polypeptide and/or protein) also encompasses derivatized peptide molecules. Briefly, in the context of the present invention, a derivative is a peptide in which one or more of the amino acid residues of the peptide have been chemically modified (for instance by alkylation, acylation, ester formation, or amide formation) or associated with one or more non-amino acid organic and/or inorganic atomic or molecular substituents (for instance a polyethylene glycol (PEG) group, a lipophilic substituent (which optionally may be linked to the amino acid sequence of the peptide by a linker residue or group such as β-alanine, γ-aminobutyric acid (GABA), L/D-glutamic acid, succinic acid, and the like), a fluorophore, biotin, a radionuclide, etc.) and may also or alternatively comprise non-essential, non-naturally occurring, and/or non-L amino acid residues, unless otherwise stated or contradicted by context (however, it should again be recognized that such derivatives may, in and of themselves, be considered independent features of the present invention and inclusion of such molecules within the meaning of peptide is done for the sake of convenience in describing the present invention rather than to imply any sort of equivalence between naked peptides and such derivatives). Non-limiting examples of such amino acid residues include for instance 2-aminoadipic acid, 3-aminoadipic acid, β-alanine, β-aminopropionic acid, 2-aminobutyric acid, 4-aminobutyric acid, 6-aminocaproic acid, 2-amino-heptanoic acid, 2-aminoisobutyric acid, 3-aminoisobutyric acid, 2-aminopimelic acid, 2,4-diaminobutyric acid, desmosine, 2,2′-diaminopimelic acid, 2,3-diamino-propionic acid, N-ethylglycine, N-ethylasparagine, hydroxylysine, allohydroxylysine, 3-hydroxyproline, 4-hydroxyproline, isodesmosine, alloisoleucine, N-methylglycine, N-methylisoleucine, 6-N-methyllysine, N-methylvaline, nor-valine, norleucine, ornithine, and statine halogenated amino acids. It is to be understood that this derivatization is not a derivatization of the present invention, but rather a derivatization already present on the growth hormone polypeptide before the purification in accordance with the method of the present invention, or a derivatization performed after purification.

### Identity

The term “identity” as known in the art, refers to a relationship between the sequences of two or more peptides, as determined by comparing the sequences. In the art, “identity” also means the degree of sequence relatedness between peptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, 3., eds., M. Stockton Press, New York, 1991; and Carillo et al., SIAM J. Applied Math. 48, 1073 (1988).

Preferred methods to determine identity are designed to give the largest match between the sequences tested. Methods to determine identity are described in publicly available computer programs. Preferred computer program methods to determine identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

For example, using the computer algorithm GAP (Genetics Computer Group, University of Wisconsin, Madison, Wis.), two peptides for which the percent sequence identity is to be determined are aligned for optimal matching of their respective amino acids (the “matched span”, as determined by the algorithm). A gap opening penalty (which is calculated as 3.times. the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp. 3 (1978) for the PAM 250 comparison matrix; Henikoff et al., Proc. Natl. Acad. Sci. USA 89, 10915-10919 (1992) for the BLOSUM 62 comparison matrix) is also used by the algorithm.

Preferred parameters for a peptide sequence comparison include the following:

Algorithm: Needleman et al., J. Mol. Biol. 48, 443-453 (1970); Comparison matrix: BLOSUM 62 from Henikoff et al., PNAS USA 89, 10915-10919 (1992); Gap Penalty: 12, Gap Length Penalty: 4, Threshold of Similarity: 0.

The GAP program is useful with the above parameters. The aforementioned parameters are the default parameters for peptide comparisons (along with no penalty for end gaps) using the GAP algorithm.

The term “similarity” is a concept related to identity, but in contrast to “identity”, refers to a sequence relationship that includes both identical matches and conservative substitution matches. If two polypeptide sequences have, for example, (fraction (10/20)) identical amino acids, and the remainder are all non-conservative substitutions, then the percent identity and similarity would both be 50%. If, in the same example, there are 5 more positions where there are conservative substitutions, then the percent identity remains 50%, but the percent similarity would be 75% ((fraction (15/20))). Therefore, in cases where there are conservative substitutions, the degree of similarity between two polypeptides will be higher than the percent identity between those two polypeptides.

Conservative modifications a peptide comprising an amino acid sequence of SEQ ID No. 1 (and the corresponding modifications to the encoding nucleic acids) will produce peptides having functional and chemical characteristics similar to those of a peptide comprising an amino acid sequence of SEQ ID No. 1. In contrast, substantial modifications in the functional and/or chemical characteristics of peptides according to the invention as compared to a peptide comprising an amino acid sequence of SEQ ID No. 1 may be accomplished by selecting substitutions in the amino acid sequence that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain.

For example, a “conservative amino acid substitution” may involve a substitution of a native amino acid residue with a normative residue such that there is little or no effect on the polarity or charge of the amino acid residue at that position. Furthermore, any native residue in the polypeptide may also be substituted with alanine, as has been previously described for “alanine scanning mutagenesis” (see, for example, MacLennan et al., Acta Physiol. Scand. Suppl. 643, 55-67 (1998); Sasaki et al., Adv. Biophys. 35, 1-24 (1998), which discuss alanine scanning mutagenesis).

Desired amino acid substitutions (whether conservative or non-conservative) may be determined by those skilled in the art at the time such substitutions are desired. For example, amino acid substitutions can be used to identify important residues of the peptides according to the invention, or to increase or decrease the affinity of the peptides described herein for the receptor in addition to the already described mutations.

Naturally occurring amino acid residues may be divided into classes based on common side chain properties:

1) hydrophobic: norleucine, Met, Ala, Val, Leu, Ile;

2) neutral hydrophilic: Cys, Ser, Thr, Asn, Gln;

3) acidic: Asp, Glu;

4) basic: His, Lys, Arg;

5) residues that influence chain orientation: Gly, Pro; and

6) aromatic: Trp, Tyr, Phe.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., J. Mol. Biol., 157, 105-131 (1982). It is known that certain amino acids may be substituted for other amino acids having a similar hydropathic index or score and still retain a similar biological activity. In making changes based upon the hydropathic index, the substitution of amino acids whose hydropathic indices are within ±0.2 is preferred, those that are within ±1 are particularly preferred, and those within ±0.5 are even more particularly preferred. The greatest local average hydrophilicity of a protein, as governed by the hydrophilicity of its adjacent amino acids, correlates with its immunogenicity and antigenicity, i.e., with a biological property of the protein.

The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those that are within ±1 are particularly preferred, and those within ±0.5 are even more particularly preferred.

Polypeptides of the present invention may also include non-naturally occurring amino acids.

### Growth Hormone Polypeptide

In the present context, the words “human growth hormone (hGH)” and “wild type hGH (wthGH)” are used interchangeably and refer both to a protein with an amino acid sequence as SEQ ID No. 1.

In context of present invention as used herein, the term “growth hormone polypeptide” means a peptide comprising an amino acid sequence, which has at least 80% identity to SEQ ID No. 1.

In one embodiment of present invention, the growth hormone polypeptide is a peptide comprising an amino acid sequence having at least 85%, such as at least 90%, for instance at least 95%, such as at least 96%, for instance at least 97%, such as at least 98%, for instance at least 99% identity to SEQ ID No. 1.

In one embodiment of present invention, the growth hormone polypeptide is a fragment of such a peptide, which fragment has retained a significant amount of the growth hormone activity, such as having substantially the same growth hormone activity, of such a peptide.

In one embodiment of the present invention a growth hormone compound is a truncated version of hGH, i.e. one or more amino acid residues have been deleted form the N- and/or C-termini corresponding to SEQ No. 1 wherein the said compound retain desired biological properties of wild type hGH.

In one embodiment of the present invention the growth hormone compound is a polypeptide comprising an amino acid sequence, which sequence is at least 20%, such as at least 30%, for instance at least 40%, such as at least 50%, for instance at least 60%, such as at least 70%, for instance at least 80%, such as at least 90% identity, for instance at least 95%, such as at least 96%, for instance at least 97%, such as at least 98%, for instance at least 99% similar to SEQ ID No. 1 and which polypeptide has an activity in relevant hGH assays of at least 1%, such as at least 5%, for instance at least 10%, such as at least 25% of the activity of hGH.

To avoid doubt, a growth hormone compound of the invention may also have a higher activity than wild type hGH. If the growth hormone compound is derivatized in some way, the activity of the growth hormone in relation to hGH should be measured on the underivatized growth hormone compound, as the derivatization may change the activity significantly. For instance in the case of a growth hormone compound derivatized with a property-modifying group that prolongs the half-life of the growth hormone compound in vivo, the activity of the derivatized growth hormone compound may be much lower than the activity of hGH, which decrease is counteracting by the prolonged residence time. In one embodiment, the growth hormone compound is a fragment of such a polypeptide, which fragment has retained a significant amount of the growth hormone activity as described above.

Other examples of GH compounds into which additional disulphide bridges may be introduced include those disclosed in WO 92/09690 (Genentech), U.S. Pat. No. 6,004,931 (Genentech), U.S. Pat. No. 6,143,523 (Genentech), U.S. Pat. No. 6,136,536 (Genentech), U.S. Pat. No. 6,057,292 (Genentech), U.S. Pat. No. 5,849,535 (Genentech), WO 97/11178 (Genentech), WO 90/04788 (Genentech), WO 02/055532 (Maxygen APS and Maxygen Holdings), U.S. Pat. No. 5,951,972 (American Cynanamid Corporation), US 2003/0162949 (Bolder Biotechnologies, Inc.) which are incorporated herein by reference.

In one currently preferred embodiment, the Growth Hormone polypeptide is a human Growth Hormone polypeptide, in particular a recombinant human Growth Hormone polypeptide. In one important variant hereof, the human Growth Hormone polypeptide is a modified human Growth Hormone polypeptide, in particular a PEGylated human Growth Hormone polypeptide, a hyperglycosylated growth hormone or a polypeptide with more than 2 disulphide bridges.

### Preparation of Affinity Resins

The affinity resins can in principle be prepared in two fundamentally different ways, namely (i) by synthesizing the ligand in free form and subsequently immobilizing the ligand to the solid phase material directly or via a linker (see above), or (ii) by functionalizing the solid phase material and thereafter sequentially synthesizing the ligand(s). With respect to the first variant, immobilization techniques are readily available in the art, e.g. in Hermanson et al. (see above). With respect to the second variant, techniques are also readily available, e.g. the techniques known in the art of solid phase peptide synthesis and derived techniques [Fields, G. B. et al. (1992) Principles and practice of solid-phase peptide synthesis. In Synthetic Peptides: A User's Guide (Grant, G. A., ed.), pp. 77-183, W.H. Freeman] and [Fields, G. B., ed. (1997) Solid-phase peptide synthesis. Methods in Enzymology 289. 3) Dorwald, F. Z. Organic synthesis on solid phase—supports, linkers, reactions; Wiley-VCH: Weinheim, 2000].

Examples hereof are provides in Examples 1 and 2.

Step (a)—Contacting the Growth Hormone Polypeptide with an Affinity Resin

In a first step of the process, the suspension or solution containing the Growth Hormone polypeptide is contacted with an affinity resin under conditions which facilitate binding of a portion of said Growth Hormone polypeptide to said affinity resin. The aim is to facilitate binding of a growth hormone containing portion of suspension of solution containing GH to said affinity resin.

By the term “portion” in connection with step (a) is meant at least 30% (i.e. 30-100%) of the mass of the Growth Hormone polypeptide present in the suspension or solution. It should be understood that it in most instances is desirable to bind far more than 30% of the mass of the Growth Hormone polypeptides, e.g. at least 50%, or at least 70%, or a predominant portion. By the term “predominant portion” is meant at least 90% of the mass of the Growth Hormone polypeptide present in the suspension or solution. Preferably an even higher portion becomes bound to the affinity resin, e.g. at least 95% of the mass, or at least 98% of the mass, or at least 99% of the mass, or even substantially all of the mass of the Growth Hormone polypeptide present in the suspension or solution containing Growth Hormone polypeptide.

The most common arrangement of the affinity resin is in a column format. Arrangement in a batch container is of course also possible.

The contacting of suspension or solution of the Growth Hormone polypeptide is typically conducted according to conventional protocols, i.e. the concentration, temperature, ionic strength, etc. of the suspension or solution may be as usual, and the affinity resin may be washed and equilibrated before application as usual.

The load of Growth Hormone polypeptide is typically at least 5.6 g per litre of affinity resin, such as in the range of 1-20.0 g, e.g. 3.0-10.0 g, Growth Hormone polypeptide per litre of affinity resin in wet form, and the suspension or solution containing Growth Hormone polypeptide is typically loaded at a flow of 1-50 column volumes per hour (CV/h), such as 25-35 CV/h.

The pH of suspension or solution containing Growth Hormone polypeptide before and upon application to the affinity resin appears to play a relevant role for the formation of contaminants, e.g. in the form of dimers and degradation products of the Growth Hormone polypeptide. Thus, it is preferred that the suspension or solution containing growth hormone polypeptide is in liquid form and has a pH in the range of 3.0-10.0, such as in the range of 3.0-7.0, or 6.5-10.0, upon application to the affinity resin. In some interesting embodiments, the suspension or solution containing growth hormone polypeptide has a pH of in the range of 4.0-7.0, or in the range of 7.0-9.0, or in the range of 4.5-8.5. A preferred pH range would be 5.0-6.5.

Typically, the conductivity is at least 1 mS/cm, such as 40 mS/cm, such as at least 50 mS/cm, such as at least 100 mS/cm such at lest 200 mS/cm.

The temperature of suspension or solution growth hormone polypeptide is typically 0-30° C., such as around 2-25° C.

The temperature of the affinity resin with the bound Growth Hormone polypeptide is typically 0-30° C., such as around 2-25° C., e.g. kept within a specified range by using a cooling jacket and solutions of controlled temperature.

Step (b)—Washing Step (Optional)

After binding Growth Hormone polypeptide to the affinity resins, a washing step (b) is typically conducted in order to remove proteins which are bound unspecific to the affinity resin. By this step, the remaining (bound) fraction of the Growth Hormone polypeptide on the affinity resin will have a much lower abundance of contaminants.

This washing step (b) is preferably conducted with a washing buffer having a pH in the range of 2.0-6.9. In some interesting embodiments, the washing buffer has a pH in the range of 3.0-10.0, such as in the range of 3.0-7.0, or 6.5-10.0, upon application to the affinity resin. In some interesting embodiments, the washing buffer has a pH of in the range of 4.0-7.0, or in the range of 7.0-9.0, or in the range of 4.5-8.5.

The washing step (b) is typically conducted at a flow of 1-50 column volumes per hour.

The washing buffer is typically an aqueous solution comprising a buffering agent, typically a buffering agent comprising at least one component selected from the groups consisting of acids and salts of MES, PIPES, ACES, BES, TES, HEPES, TRIS, BISTRIS, triethanolamine, histidine, imidazole, glycine, glycylglycine, glycinamide, phosphoric acid, acetic acid (e.g. sodium acetate), lactic acid, glutaric acid, citric acid, tartaric acid, malic acid, maleic acid, and succinic acid. It should be understood that the buffering agent may comprise a mixture of two or more components, wherein the mixture is able to provide a pH value in the specified range. As examples can be mentioned acetic acid and sodium acetate, etc.

In addition to a buffering agent, the washing buffer may also contain non-ionic detergents such as NP40, Triton-X100, Tween-80, or other additives such as caprylic acid.

In addition to a buffering agent, the washing buffer may also contain ionic strength increasing agents that do not change the pH of the buffer, such as sodium chloride, sodium sulphate and the like.

In one embodiment of present invention, step (b) involves at least one washing buffer comprising 0-50 mM BisTris at pH 6.0-6.5, preferably at around room temperature.

In one embodiment of present invention, step (b) involves at least one washing buffer comprising 25 mM Tris-HCl at pH 7.5 (buffer A)

It should be understood that the washing step (b) may be conducted by using one, two or several different washing buffers, or by the application of a gradient washing buffer.

It should also be noted that the washing step and the elution step need not to be discrete steps, but may be combined, in particular if a gradient elution buffer is utilised in the elution step.

Step (c)—Elution Step

After the washing step(s) (c), the affinity resin is eluted with an elution buffer, and a purified Growth Hormone polypeptide is collected as an eluate.

A great deal of variability is possible for the elution step (c).

The type of elution is not particularly critical, thus, it is, e.g., possible to elute with an elution buffer comprising a stepwise decreasing gradient of salts, elute with a linear decreasing gradient of the salts (or a gradient-hold-gradient profile, or other variants), or to use a pH gradient, or to use a temperature gradient, or a combination of the before-mentioned.

The conductivity of the final elution buffer is preferably higher than the conductivity of the composition comprising the growth hormone polypeptide in step (a).

In most instances, the elution buffer in step (c) typically has a pH as in step (a) and (b). However, the elution buffer in step (c) may also have a pH higher than in step (a) and (b).

Also preferred are the embodiments where the elution buffer in step (c) has a pH between 7.0 and 8.0.

In an even more preferred embodiment, the elution buffer comprises 25-200 mM BisTris at pH 6.5-7.5, preferably at around room temperature.

In another preferred embodiment, the elution buffer comprises 25-200 mM TRIS at pH 7.5-8.0.

In another preferred embodiment, the elution buffer comprises 50 mM Triethanolamine at pH 8.0.

In another preferred embodiment, the elution buffer comprises 1 M NaCl or 1 M MgCl₂ in combination with either of the above mentioned buffers.

In another preferred embodiment, the elution buffer contains 5-30% v/v glycerol or propylene glycol, in combination with either of the above mentioned buffers.

Typically, the process of the present invention is capable reducing the content of other proteins with at least 50%, however more preferably, and also realistically, the reduction is at least 60%, such as at least 70% or even at least 80% or at least 85%.

Usually, the affinity resin can be regenerated for the purpose of subsequent use by a sequence of steps.

It should be noted that the washing step and the elution step need not to be discrete steps, but may be combined, in particular if a gradient elution buffer is utilised in the elution step.

Although not limited thereto, the process of the present invention is particularly feasible for “industrial-scale” (or “large-scale”) purification of a Growth Hormone polypeptide. By the term “industrial-scale” is typically meant methods wherein the volume of liquid Growth Hormone polypeptide compositions is at least 10 L, such as at least 50 L, e.g. at least 500 L, or at least 5000 L, or where the weight of the product is at least 10 g (dry matter), such as at least 100 g, e.g. at least 500 g, e.g. 1-15,000 g.

### Novel Affinity Resins

It is believed that some of the most interesting affinity resins described herein are novel as such. Hence, the present invention also provides novel affinity resins comprising a solid phase material having covalently immobilized there to one or more ligands, i.e. the ligands described hereinabove.

The present invention also provides novel affinity resins comprising a solid phase material having covalently immobilized there to one or more ligands, i.e. the ligands described hereinabove.

One embodiment of present invention, provides for a process for the purification of Growth Hormone polypeptide, said process comprising the steps of:

(a) contacting a suspension or solution containing growth hormone polypeptide with an affinity resin under conditions which facilitate binding of a portion of said growth hormone polypeptide to said affinity resin;

(b) optionally washing said affinity resin containing growth hormone polypeptide with a washing buffer; and

(c) eluting said affinity resin containing growth hormone polypeptide with an elution buffer, and collecting a Growth Hormone polypeptide as an eluate;

wherein said affinity resin comprising a solid phase material having covalently immobilized thereto one or more ligands of the general formula (I),

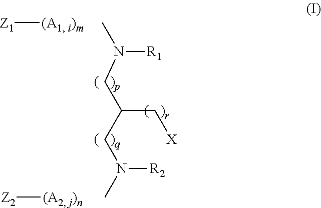

wherein

i=1, 2, . . . , m, and j=1, 2, . . . , n;

n and m are independently an integer in the range of 0-3, with the proviso that the sum n+m is in the range of 1-4;

p, q, and r are independently an integer in the range of 0-6;

A11, . . . , A1m and A21, . . . , A2n are independently selected from α-amino acid moieties, β-amino acid moieties, α-amino sulphonic acid moieties, and β-amino sulphonic acid moieties;

Z1 and Z2 are independently selected from hydrogen, C₁₋₆ alkyl, carboxylic acid moieties (Z—C(═O)—), and sulphonic acid moieties (Z—S(═O)₂—), wherein Z is selected from hydrogen, optionally substituted C₁₋₁₂-alkyl, optionally substituted C₃₋₁₂-cycloalkyl, optionally substituted C₁₋₁₂-alkenyl, optionally substituted C₁₋₁₂-alkynyl, optionally substituted aryl, optionally substituted heteroaryl and optionally substituted heterocyclyl;

R1 and R2 are independently selected from hydrogen and C₁₋₆-alkyl;

X is the group for attachment of the ligand to the solid phase material, either directly or via a linker, X being selected from carboxylic acid (—COOH), a carboxylic acid ester (—COOR), a carboxylic acid anhydride (—COOCOR), a carboxylic acid halide (—COHal), sulphonic acid (—S(═O)₂OH), a sulphonyl chloride (—S(═O)₂Cl), thiol (—SH), a disulphide (—S—S—R), hydroxy (—OH), aldehyde (C(═O)H), epoxide (—CH(O)CH₂), cyanide (—CN), halogen (-Hal), primary amine (—NH₂), secondary amine (—NHR), hydrazide (—NH═NH₂), and azide (—N₃), wherein R is selected from optionally substituted C₁₋₁₂-alkyl, and Hal is a halogen; and

the total molecular weight of said ligand (excluding “X” and any linker) being 200-2000 g/mol.

In one embodiment of present invention, Z1 and Z2 are independently selected from hydrogen, C₁₋₆ alkyl, xanthene-9-carbonyl, 2-amino nicotinyl, 2-quinaldincarbonyl, 4,8-dihydroxy-2-quinolinecarbonyl, 4-quinolinecarbonyl, 5-methyl-2-nitrobenzoyl, 2-(benzoimidazolylthio)acetyl, 5-methyl-2-phenyl-2H-1,2,3-triazole-4-carbonyl, 6-hydroxy-2-naphthoyl, 4,7-dimethylpyrazolo[5,1-c][1,2,4]triazine-3-carbonyl, 3-amino-4-(phenylsulfonyl)-2-thiophenecarbonyl, (+/−)-3-oxo-1-indancarbonyl, 5,6,7,8-tetrahydroacridine-9-carbonyl, 2-methylimidazo[1,2-a]pyridine-3-carbonyl, 5-(4-methyl-2-nitrophenyl)furoyl, 1-cyclohexyl-4-oxo-1,4-dihydroquinoline-3-carbonyl, quinoxaline-6-carbonyl, and 4-methyl-2-phenylpyrimidine-5-carbonyl.

In one embodiment of present invention, Z1 and Z2 are independently selected from Z1 and Z2 are independently selected from hydrogen, C₁₋₆ alkyl, xanthene-9-yl-carbonyl, 5-methyl-2-phenyl-2H-1,2,3-triazole-4-yl-carbonyl, 3-amino-(phenylsulfonyl)-thiophen-2-yl-carbonyl, (+/−)-3-oxo-1-indanyl, 5,6,7,8-tetrahydroacridine-9-yl-carbonyl, and 2-methylimidazo[1,2-a]pyridine-3-yl-carbonyl.

In one embodiment of present invention, Z1 comprises a tricyclic optionally substituted heteroaromatic group.

In one embodiment of present invention, each of Z1-(A1i)ₘ-N(R1)- and Z2-(A2i)ₙ-N(R2)- represents an organic moiety of a molecular weight of 50-500 g/mol, wherein the total molecular weight of the ligand is 250-1500 g/mol, such as 300-1200 g/mol, e.g. 350-1000 g/mol.

In one embodiment of present invention, the ligands are as specified hereinabove for general formulae (I), (II) and (III) and further in accordance with the various embodiments, in particular those embodiments of the general formulae (II) and (III). The currently most interesting ligands are ligands Nos. (1)-(16) illustrated above.

In one embodiment of present invention, provides a process for the purification of Growth Hormone polypeptide, said process comprising the steps of:

(a) contacting a suspension or solution containing growth hormone polypeptide with an affinity resin under conditions which facilitate binding of a portion of said growth hormone polypeptide to said affinity resin;

(b) optionally washing said affinity resin containing growth hormone polypeptide with a washing buffer; and

(c) eluting said affinity resin containing growth hormone polypeptide with an elution buffer, and collecting a Growth Hormone polypeptide as an eluate;

wherein ligand has the general formula (II),

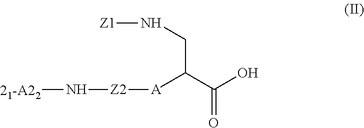

wherein

Z1 is Z—C(═O)—, wherein Z is selected from optionally substituted aryl, optionally substituted heteroaryl and optionally substituted heterocyclyl;

Z2 is selected from hydrogen, and Z—C(═O)—, wherein Z is selected from optionally substituted C₁₋₁₂-alkyl, optionally substituted C₃₋₁₂-cycloalkyl, optionally substituted aryl, optionally substituted heteroaryl and optionally substituted heterocyclyl; and

each of A2₁ and A2₂ is independently selected from α-amino acids and β-amino acids.

In one embodiment of present invention provides for a, wherein A2₁ is selected from arginine, phenylalanine, tyrosine, isoleucine, and lysine, and A2₂ is selected from arginine, phenylalanine, isoleucine, proline, tyrosine, and tryptophan.

In one embodiment of present invention provides a process of purification of Growth hormone polypeptide wherein the ligand has the general formula (III),

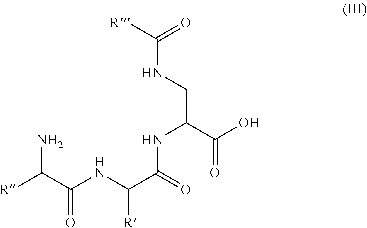

wherein R′ and R″ are independently selected from side chains of α-amino acids, and R″′ is selected from the group consisting of optionally substituted aryl, optionally substituted heteroaryl and optionally substituted heterocyclyl.

In one embodiment of present invention provides for a process according, wherein said ligand is selected from Nos. (1)-(16):

| No. | Structure |
|---|---|
| 1 | 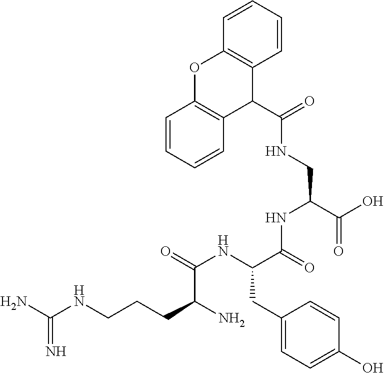 |
| 2 | 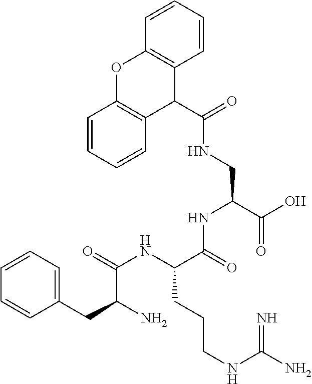 |
| 3 | 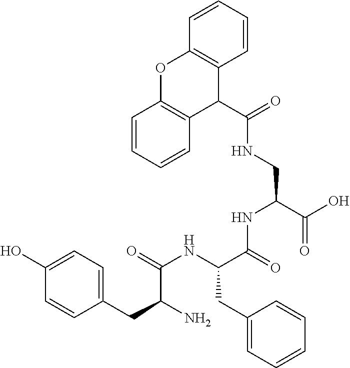 |
| 4 | 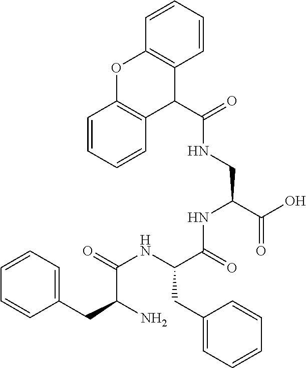 |
| 5 | 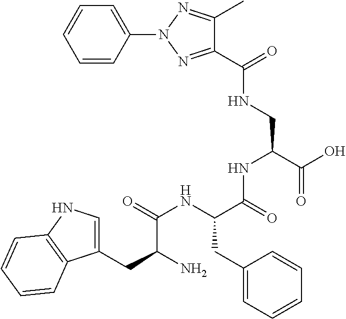 |
| 6 | 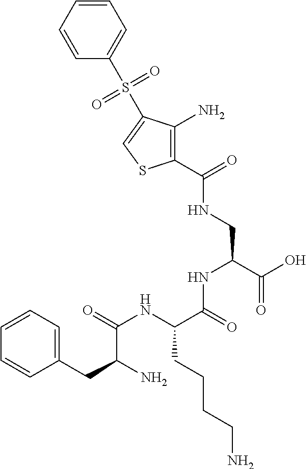 |
| 7 | 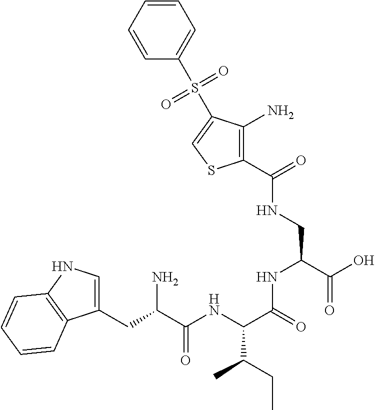 |
| 8 | 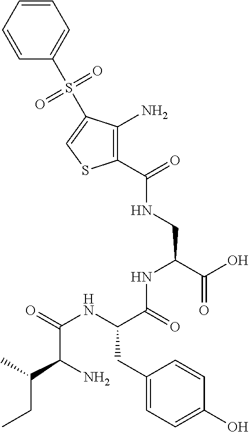 |
| 9 | 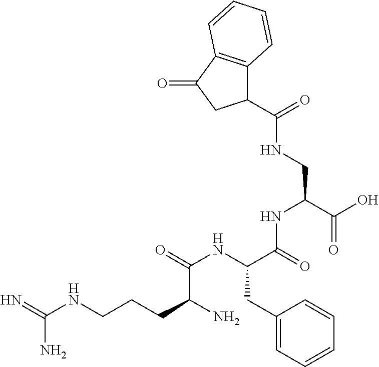 |
| 10 | 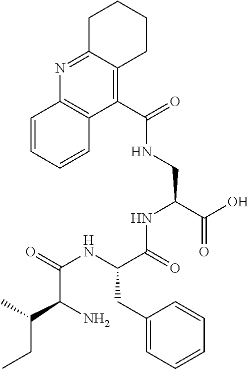 |
| 11 | 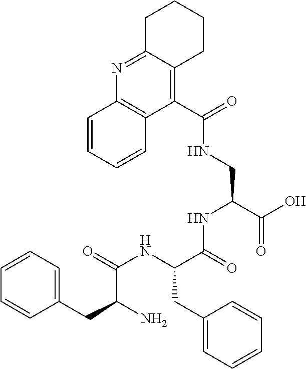 |
| 12 | 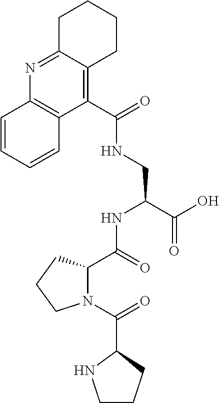 |
| 13 | 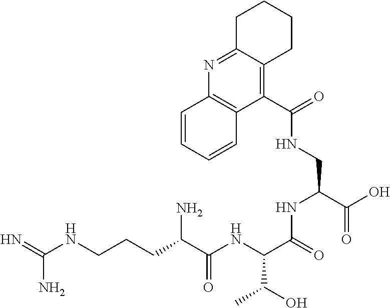 |
| 14 | 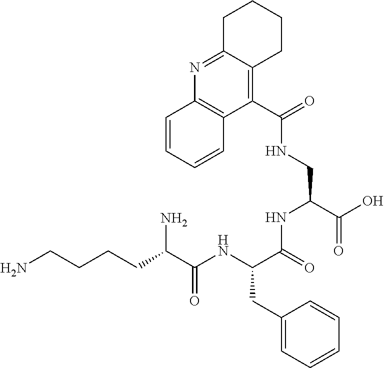 |
| 15 | 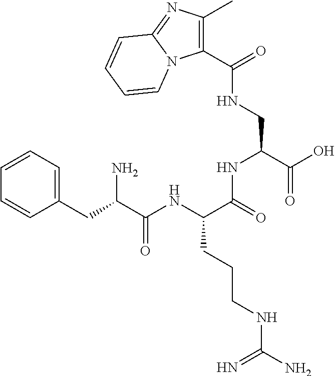 |
| 16 | 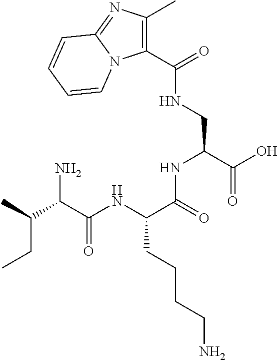 |

One embodiment of present invention provides a process of purification of Growth Hormone polypeptide wherein, in step (b), at least one washing buffer comprising 0-50 mM BisTris at pH 6.0-6.5.

One embodiment of present invention provides a process of purification of Growth Hormone polypeptide wherein, in step (c), the elution buffer has a pH between 7.0 and 8.0.

One embodiment of present invention provides a process of purification of Growth Hormone polypeptide wherein, the elution buffer comprises 0-200 mM BisTris at pH 7.0-7.5.

One embodiment of present invention provides a process of purification of Growth Hormone polypeptide wherein, the Growth Hormone polypeptide is a human Growth Hormone polypeptide.

One embodiment of present invention provides a process of purification of Growth Hormone polypeptide wherein, the human Growth Hormone polypeptide is a recombinant human Growth Hormone polypeptide.

One embodiment of present invention provides a process of purification of Growth Hormone polypeptide wherein, the human Growth Hormone polypeptide is a modified human Growth Hormone polypeptide.

One embodiment of present invention provides a process of purification of Growth Hormone polypeptide wherein, the modified human Growth Hormone polypeptide is a PEGylated human Growth Hormone polypeptide.

One embodiment of present invention provides an affinity resin comprising a solid phase material having covalently immobilized thereto one or more ligands of the formula (I)

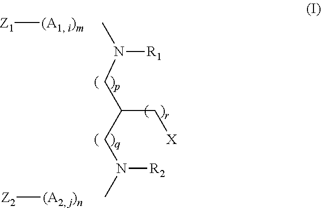

wherein

i=1, 2, . . . , m, and j=1, 2, . . . , n;

n and m are independently an integer in the range of 0-3, with the proviso that the sum n+m is in the range of 1-4;

p, q, and r are independently an integer in the range of 0-6;

A11, . . . , A1m and A21, . . . , A2n are independently selected from α-amino acid moieties, β-amino acid moieties, α-amino sulphonic acid moieties, and β-amino sulphonic acid moieties;

Z1 and Z2 are independently selected from hydrogen, C₁₋₆ alkyl, carboxylic acid moieties (Z—C(═O)—), and sulphonic acid moieties (Z—S(═O)₂—), wherein Z is selected from hydrogen, optionally substituted C₁₋₁₂-alkyl, optionally substituted C₃₋₁₂-cycloalkyl, optionally substituted C₁₋₁₂-alkenyl, optionally substituted C₁₋₁₂-alkynyl, optionally substituted aryl, optionally substituted heteroaryl and optionally substituted heterocyclyl;

R1 and R2 are independently selected from hydrogen and C₁₋₆-alkyl;

X is the group for attachment of the ligand to the solid phase material, either directly or via a linker, X being selected from carboxylic acid (—COOH), a carboxylic acid ester (—COOR), a carboxylic acid anhydride (—COOCOR), a carboxylic acid halide (—COHal), sulphonic acid (—S(═O)₂OH), a sulphonyl chloride (—S(═O)₂Cl), thiol (—SH), a disulphide (—S—S—R), hydroxy (—OH), aldehyde (C(═O)H), epoxide (—CH(O)CH₂), cyanide (—CN), halogen (-Hal), primary amine (—NH₂), secondary amine (—NHR), hydrazide (—NH═NH₂), and azide (—N₃), wherein R is selected from optionally substituted C₁₋₁₂-alkyl, and Hal is a halogen; and

the total molecular weight of said ligand (excluding “X” and any linker) being 200-2000 g/mol.

One embodiment of present invention provides for affinity resins, wherein the ligand is as specified according to any one of the above embodiments.

One embodiment of present invention provides for ligands selected from the group consisting of ligands (1)-(16) as described above, wherein the ligand is covalently attached to the solid phase material of said affinity resins via the carboxylic acid group, either directly or via a linker.

One embodiment of present invention provides for affinity ligands selected from the group consisting of ligands (1)-(16) defined herein.

The novel affinity resins are particularly useful in the purification and/or isolation of biomolecules, such as proteins, in particular Growth Hormone polypeptides. The affinity ligands are specific binding partners for Growth Hormone polypeptides and can isolate said polypeptide from closely related proteins.

In one embodiment of present invention, the ligand is immobilized to the surface of a sensor or an array plate (the “solid phase material”) and is used to detect and/or quantify Growth Hormone polypeptides in a biological sample.

When used herein, the term “biological sample” includes natural samples or samples obtained from other processes, e.g. industrial processes, recombinant processes, and include “body fluid”, i.e. any liquid substance extracted, excreted, or secreted from an organism or tissue of an organism. A body fluid need not necessarily contain cells. Body fluids of relevance to the present invention include, but are not limited to, whole blood, serum, urine, plasma, cerebral spinal fluid, tears, milk, sinovial fluid, and amniotic fluid.

In one embodiment of present invention, a plurality of ligands are immobilized to the surface of an array plate (the “solid phase material”) and arranged in a plurality of spots, with each spot representing one ligand. Such a functionalized array can be used to detect the presence of Growth Hormone polypeptides in a solution. Such an array can be used for diagnostic applications to detect the presence of Growth Hormone polypeptides in a biological sample.

In one embodiment of present invention, a plurality of ligands are immobilized to the binding surface of a cantilever sensor (the “solid phase material”) for detection and optionally quantification of Growth Hormone polypeptides. A plurality of affinity ligands can be immobilized to a plurality of cantilevers with each cantilever representing one ligand. Such a functionalized array can be used to detect the presence of various antibodies in a solution. Such a multi-sensor can be used for diagnostic applications to detect the presence of certain Growth Hormone polypeptides in a biological sample.

Furthermore, it is believed that some of the ligands are novel as such.

Hence, the invention further provides affinity ligands as specified above with general formulae (I), (II) and (III), in particular those selected from the group consisting of ligands (1)-(16).

### EXAMPLES

### Example 1

Development of a small-molecule affinity resin for the purification of human growth hormone (hGH), using a solid-phase combinatorial approach along with encoded beads technology.

hGH is a protein hormone stimulating growth and cell reproduction in humans. It binds its receptor, hGHbp, by forming an active 1:2 (hGH:hGHbp) complex. Although the hormone binds the same site on its receptor, the two binding sites on hGH are structurally distinct with Site 1 having the highest affinity. Site 1 is a large protein surface, encompassing more than 30 amino acids on each protein. The affinity is concentrated on a few residues, particularly Trp104 and Trp169 of the receptor.

### In Silico Screening and Library Design.

To construct a small-molecule mimic of the natural ligand, a branched structure (IV) with 3 points of diversity was selected.

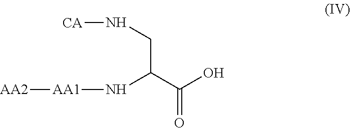

where AA2 and AA1 are amino acid residues, and CA is a carboxylic acid residue. To increase the likelihood of finding active ligands, a virtual 40,000 compound library was screened in silico against a crystal structure of hGH (1A22). The library was designed by selecting a set of 2,500 building blocks from a chemicals database (ACD). The library was constructed in Sybyl (Legion) by incorporating building blocks in the R1, R2, and R3-positions. Docking was likewise done in Sybyl using the docking algorithm FlexX. From these results, a subset of 18 building blocks were chosen for CA and 11 building blocks for each of AA1 and AA2 based on several different scoring functions as well as on chemical stability, toxicity, and availability.

AA1 and AA2 were independently selected from Asp, His, Asn, Thr, Pro, Trp, Lys, Tyr, Ile, Phe, and Arg. CA was selected from, CA1-CA18 as represented in Table I

TABLE I

| No. | Carboxylic Acid (CA) | Structure |
|---|---|---|
| CA1 | Xanthene-9- carboxylic acid | 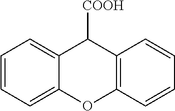 |
| CA2 | 2-Aminonicotinic acid | 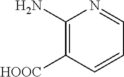 |
| CA3 | 2-Quinaldinic acid | 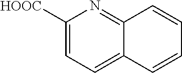 |
| CA4 | Xanthurenic acid | 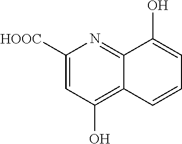 |
| CA5 | 4-Quinaldinic acid | 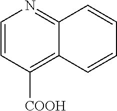 |
| CA6 | 5-Methyl-2- nitrobenzoic acid | 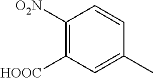 |
| CA7 | 2-(Benzoimidazolythio) acetic acid | 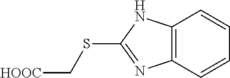 |
| CA8 | 5-Methyl-2-phenyl- 2H-1,2,3-triazole- 4-carboxylic acid | 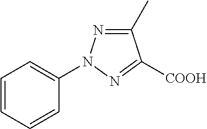 |
| CA9 | 6-Hydroxy-2- naphthoic acid | 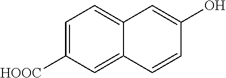 |
| CA10 | 4,7-Dimethylpyrazolo [5,1-c][1,2,4]triazine- 3-carboxylic acid | 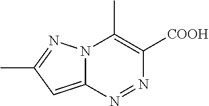 |
| CA11 | 3-Amino-4- (phenylsulfonyl)- 2-thiophenecarboxylic acid | 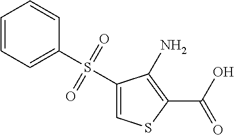 |
| CA12 | (+/−)-3-Oxo-1- indancarboxylic acid | 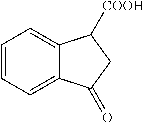 |
| CA13 | 5,6,7,8- Tetrahydroacridine- 9-carboxylic acid | 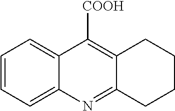 |
| CA14 | 2-Methylimidazo[1,2-a] pyridine-3-carboxylic acid | 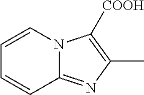 |
| CA15 | 5-(4-Methyl-2- nitrophenyl) furoic acid | 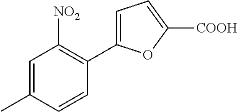 |
| CA16 | 1-Cyclohexyl-4- oxo-1,4- dihydroquinoline- 3-carboxylic acid | 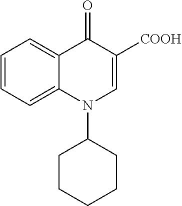 |
| CA17 | Quinoxaline-6- carboxylic acid | 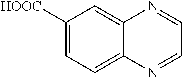 |
| CA18 | 4-Methyl-2- phenylpyrimidine- 5-carboxylic acid | 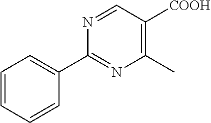 |

### Library Synthesis and Screening

The ligand library was synthesized by split-and-mix solid-phase synthesis on encoded polyethyleneglycol-acrylamide (PEGA) beads. The full library consisted of 2178 unique compounds. The bead-encoding technique is based on 3-dimensional image recognition of patterns made by fluorescent particles immobilized in random positions inside PEGA-beads. The individual bead-codes are recorded after each chemical transformation by an instrument equipped with 3 orthogonal CCD cameras. The three images are triangulated to give each bead a unique code, enabling its chemical history to be tracked [S. F. Christensen, M. Meldal, Genetic Engineering News, 25, No. 7, Apr. 1, 2005].

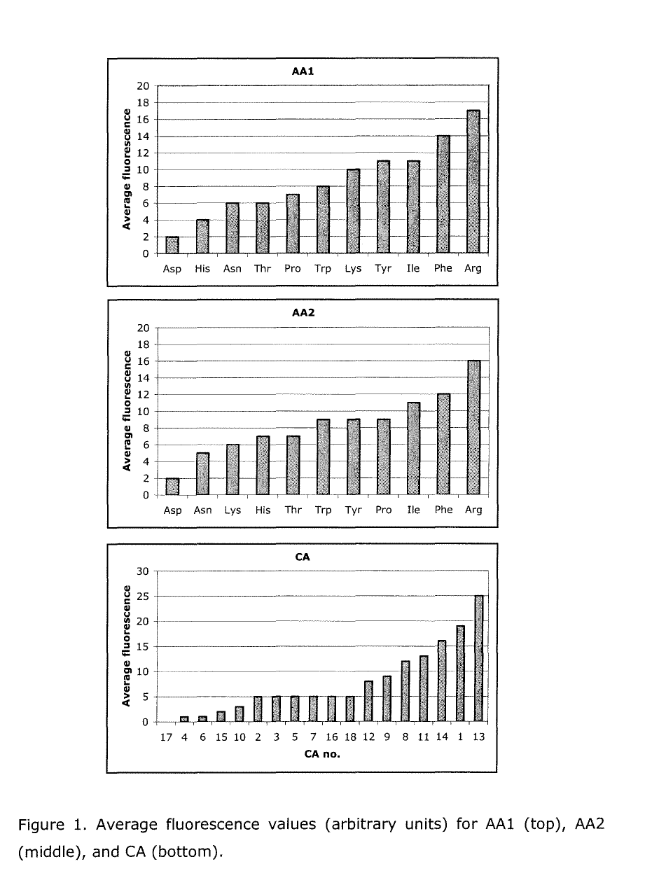

The beads were incubated with Rhodamine-labeled hGH in PBS, washed with PBS, and their fluorescence measured and quantified. A bead with a ligand with high affinity for hGH has a high fluorescence value, and a bead with a ligand with low affinity for hGH has a low fluorescence. By matching the fluorescence value of every bead with the building block sequence of the ligand it carries the structure-affinity relationship was established for all 2178 compounds. The fluorescence value for each compound is not shown here. Instead, average fluorescence values for each of AA1, each of AA2, and each of CA are shown in FIG. 1.

Based on the structure-affinity data, 16 expected high-affinity ligands are represented in Table II (L1-L16),

TABLE II

| | | Measured | |
|---|---|---|---|
| | | binding capacity | |
| | | (mg hGH/mL | Observed |
| No. | Structure | resin) | selectivity |
| L1 | 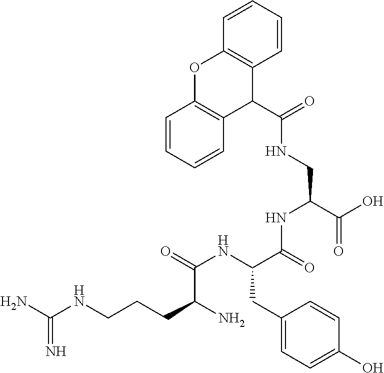 | 14 | Moderate |
| L2 | 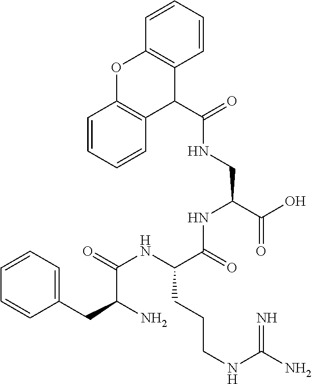 | 7 | Good |
| L3 | 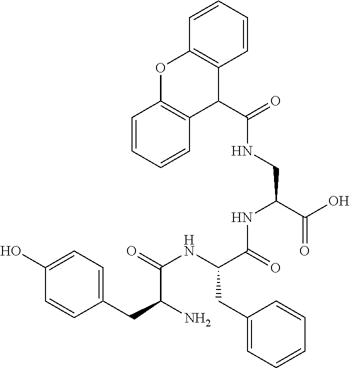 | 5 | Moderate |
| L4 | 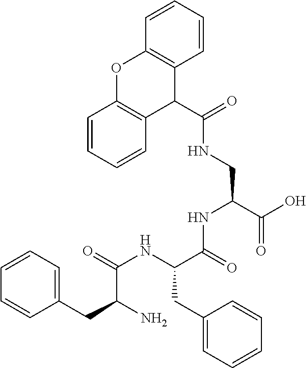 | 1 | Good |
| L5 | 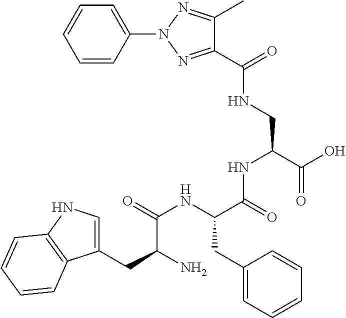 | 2 | Good |
| L6 | 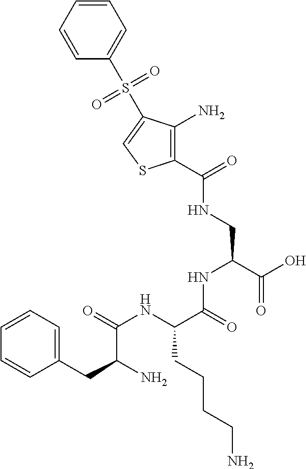 | 13 | Moderate |
| L7 | 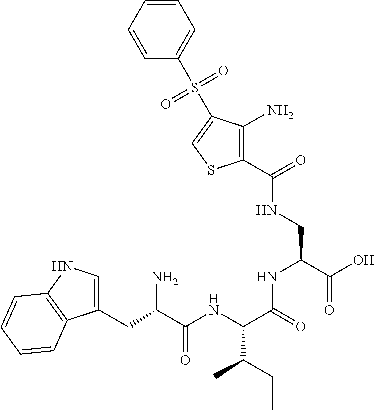 | 2 | Good |
| L8 | 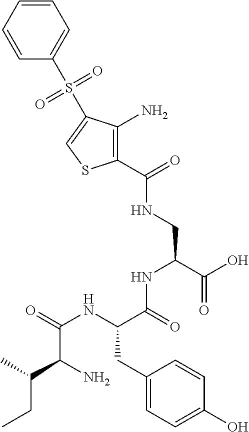 | 6 | Moderate |
| L9 | 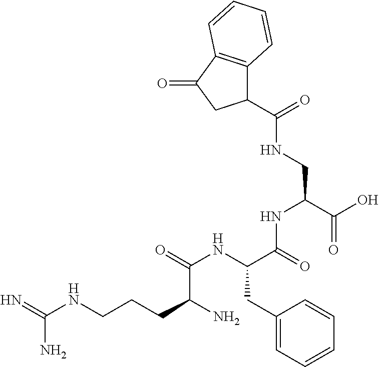 | 10 | Good |
| L10 | 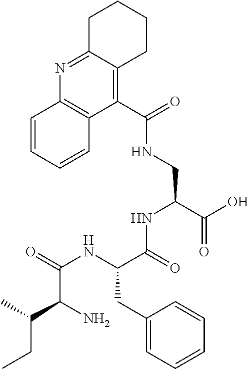 | 3 | Very good |
| L11 | 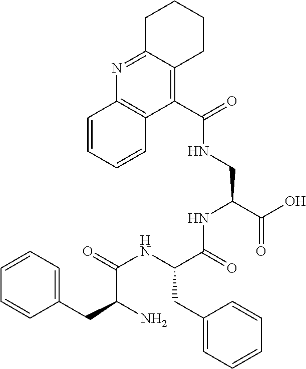 | 2 | Very good |
| L12 | 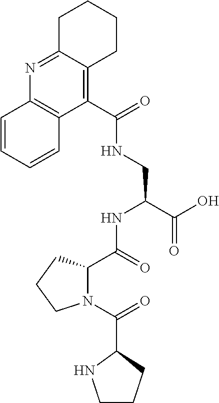 | 2 | Good |
| L13 | 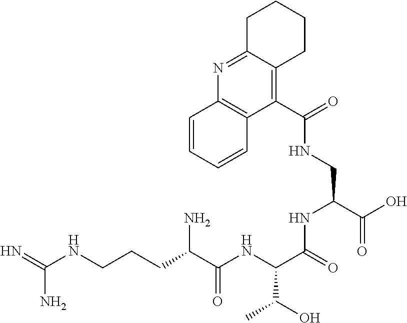 | >10 | Moderate |
| L14 | 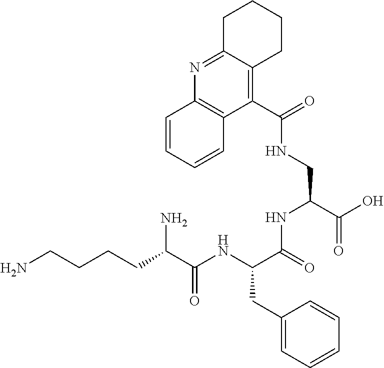 | >5 | Good |
| L15 | 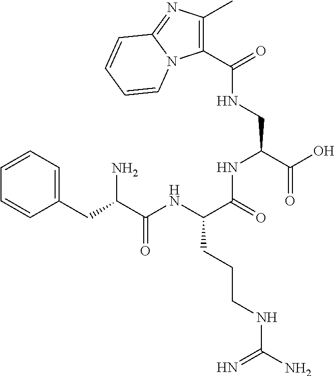 | >5 | Good |
| L16 | 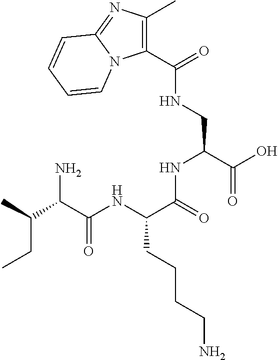 | >10 | Moderate |

and two expected low affinity ligands are represented in Table III (No. L17-L18),

TABLE III

| | | Bind- | |
|---|---|---|---|
| | | ing | |
| | | capa- | |
| | | city | |
| | | (mg | Se- |
| | | hGH/ | lec- |
| | | mL | tiv- |
| No. | Structure | resin) | ity |
| L17 | 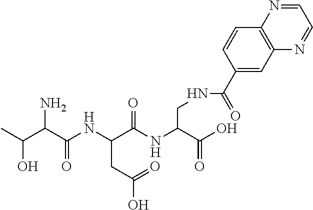 | <0.5 | N/A |
| L18 | 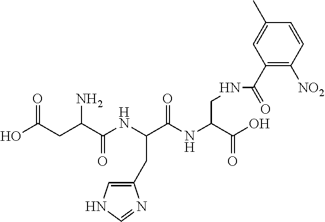 | <0.5 | N/A |

were stepwise synthesized on Fractogel Amino (Merck) with amine loading 0.16 mmol/g and particle size 40-90 μm. The resins were washed thoroughly with DMF, DCM, and ethanol and packed in 1 mL columns and washed with 0.2 M NaOH and then with 20% ethanol, and equilibrated with 25 mM Tris-HCl at pH=7.50 (buffer A).

Binding capacity for wild type hGH was measured for each resin sample by
a) loading the column with a 5 mg/mL solution of hGH in buffer A at a flow rate of 0.5 mL/min,
b) washing the column with at least 10 column volumes of buffer A until a steady low UV absorbance of the buffer leaving the column was observed,
c) eluting the protein by applying a gradient of 1M NaCl/25 mM Tris at pH=7.5 (buffer B)
d) cleaning the column with 0.2 M NaOH, then with buffer B, and then with buffer A.

The binding capacity was calculated by integrating the UV signal during step (c) and indicated for each ligand above.

Resin selectivity was tested by
(a) loading the column with hGH fermentation broth diluted five times with buffer A
(b) washing the column with at least 10 column volumes of buffer A until a steady low UV absorbance of the buffer leaving the column was observed,
(c) eluting the protein by applying a gradient of buffer B
(d) cleaning the column with 0.2 M NaOH, then with buffer B, and then with buffer A.

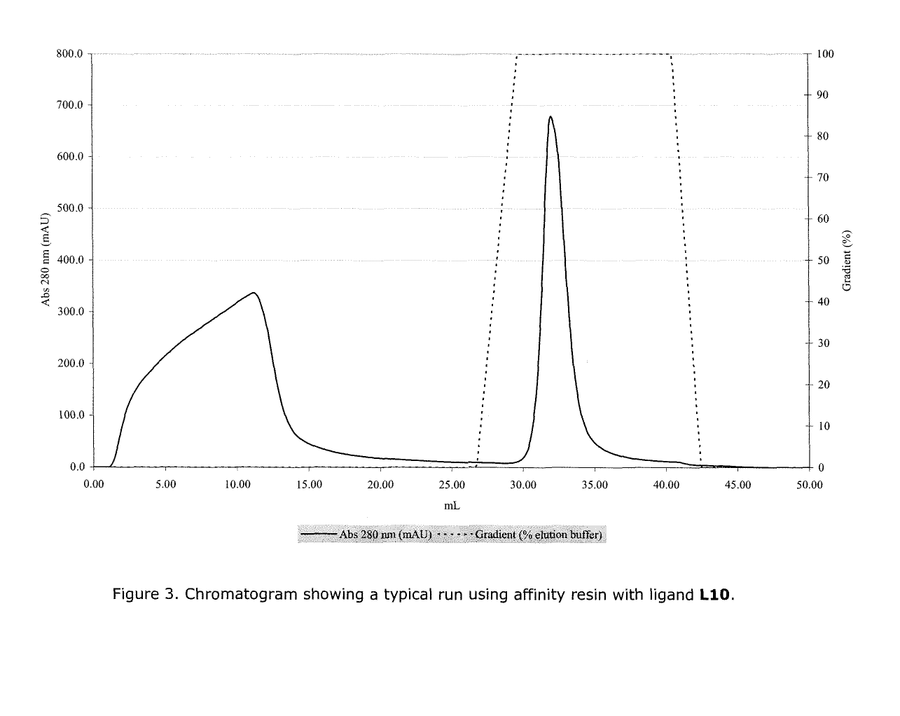

“Observed selectivity” was judged by SDS page gels of the eluate obtained during step (g) and indicated for each ligand above. An example of a resin with “very good” selectivity (ligand L11 on Fractogel) and an example of a resin with “moderate” selectivity (ligand L6 on Fractogel) are given in FIG. 3.

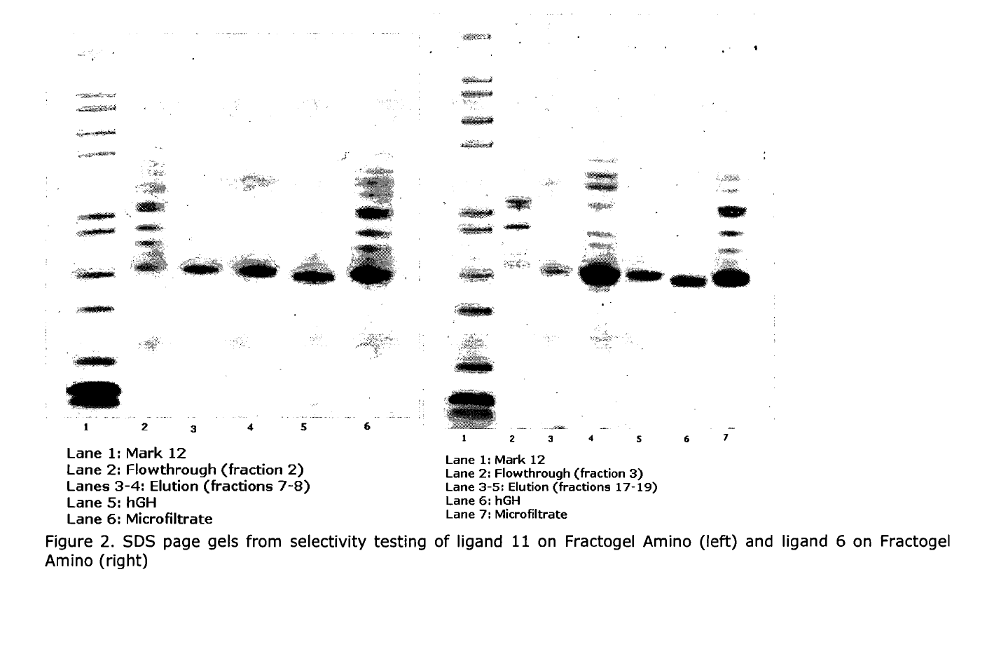

FIG. 2. SDS page gels from selectivity testing of ligand 11 on Fractogel Amino (left) and ligand 6 on Fractogel Amino (right).

### Example 2

A ligand with structure,

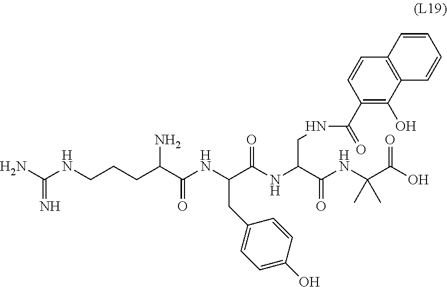

was synthesized on Fractogel Amino and tested by the same procedures as described in Example 1. The resulting resin had a binding capacity of <0.5 mg/mL. The selectivity was not tested.

The carboxylic acid residue of L19 is a substituted napthoyl and structurally resembles CA3, CA4, CA5, CA9 and CA17 in Example 1, all of which result in very low to moderate average fluorescence values according to FIG. 2. On this basis one would expect L19 to have low affinity towards hGH. On the other hand, according to Table 1 one would expect the amino acid residue combination (AA1, AA2)=(Tyr, Arg) of L19 to give rise to high affinity towards hGH. However, it appears that the expected positive effect of the amino acid residue combination on the ligands affinity towards hGH is off-set by the expected negative effect of the carboxylic acid residue resulting in a net low hGH affinity of L19.

### Example 3

### Direct Synthesis of Ligand L10 from Example 1 on Amino Functionalized Fractogel Resin

Fractogel EMD-amino resin (70 mL, 2.34 mmol, supplied by Merck KGaA) was washed with water (3×), EtOH (3×), and DMF (5×) in a fritted syringe and transferred to a round-bottomed flask (250 mL). Fmoc-L-DAPA(Alloc)-OH (2.88 g, 3.0 eq, 7.0 mmol) and TBTU (2.10 g, 2.8 eq, 6.5 mmol) were dissolved in DMF (50 mL) and N-ethylmorpholine (1.18 mL, 4.0 eq, 9.4 mmol) and preactivated for 10 min. The clear solution was added to the resin and additional 50 mL of DMF was added. The flask was placed on a shaker overnight. The resin was transferred to a large fritted syringe and washed with DMF (5×) and DCM (5×). A loading of 0.19 mmol/g was determined by Fmoc-quantification of a small sample. Remaining amino-residues were capped with 20% acetic anhydride in DMF for 1 h. The resin was washed with DMF (5×) and DCM (5×). A negative Kaiser test indicated the absence of free amino groups on the resin. A portion of the resin (15 mL) was washed with DMF (×5) and the Fmoc protecting group was removed by treatment with 20% piperidine in DMF for 2+30 min. The resin was washed with DMF (5×) and DCM (×5) and a small sample gave a positive Kaiser test. The resin was washed with DMF (5×). Fmoc-L-Phe-OH (582 mg, 3.0 eq, 1.50 mmol) and TBTU (450 mg, 2.8 eq, 1.40 mmol) were dissolved in DMF (10 mL) and NEM (254 μL, 4.0 eq, 2.00 mmol) and preactivated for 10 min. The solution was added to the resin and shaken overnight in a capped fritted syringe. The resin was washed thoroughly with DMF (×5), DCM (×5) and gave a negative Kaiser test. The resin was washed with DMF (×5) and the Fmoc protecting group removed by treatment with 20% piperidine in DMF for 2+30 min. The Kaiser test was positive. The resin was washed with DMF (5×). Fmoc-L-Ile-OH (532 mg, 3.0 eq, 1.50 mmol) and TBTU (450 mg, 2.8 eq, 1.40 mmol) were dissolved in DMF (10 mL) and NEM (254 μL, 4.0 eq, 2.00 mmol) and preactivated for 10 min. The solution was added to the resin and shaken overnight in a capped fritted syringe. The Kaiser test was negative but with a slight blueish tint. The resin was capped with 20% acetic anhydride in DMF for 1 h and washed with DMF (5×), DCM (5×) to give a negative Kaiser test. The syringe holding the resin was fitted with a rubber septum and kept under an atmosphere of N₂. The resin was washed with dry DCM (2×) that had been thoroughly degassed with N₂. To remove the Alloc protecting group, Me₂NH.BH₃ (590 mg, 20 eq, 10.0 mmol) was dissolved in dry degassed DCM (10 mL) and added to the resin which was shaken and left standing for 10 min. Pd(PPh₃)₄ (116 mg, 0.2 eq, 0.1 mmol) was dissolved in degassed DCM (1 mL) and added to the resin which was shaken for 1 h. The resin was washed with DCM (3×) and the procedure repeated to ensure full deprotection. The resin was thoroughly washing with DCM (5×), DMF (5×), DCM (5×), EtOH (5×), DCM (5×), and DMF (5×). 1,2,3,4-Tetrahydroacridine-9-carboxylic acid (342 mg, 3.0 eq, 1.50 mmol) and TBTU (450 mg, 2.8 eq, 1.40 mmol) were dissolved in DMF (10 mL), NEM (254 μL, 4.0 eq, 2.00 mmol) and 10 drops of DMSO and preactivated for 10 min. The solution was added to the resin and shaken overnight in a capped fritted syringe. The resin was washed with DMF (5×), DCM (5×), and DMF (5×) and the coupling procedure repeated as above. The Fmoc protecting group was removed by treatment with 20% piperidine in DMF for 5+40 min. Ligands bearing acid-labile sidechain protecting groups were treated with TFA/water/TIS (93:5:2) for 1 h. The resin was washed thoroughly with DCM, EtOH, DCM, DMF, 5% DIPEA in DMF, DMF, DCM, EtOH, and water.

### Example 4

### Synthesis of Ligand L10 from Example 1 and Coupling to Amino Functionalized Fractogel Resin

Ligand L10 was synthesized in the manner described in Example 1 on an aminomethyl polystyrene resin (supplied by CBL Patras) using an acid cleavable 4-hydroxymethylphenoxyacetic acid (HMPA, 3 eq.) linker attached using DIC (3 eq.) and HOBt (3 eq.) in DCM. Fmoc-L-DAPA(Alloc)-OH (3 eq) was attached to the resin using 1-(mesitylene-2-sulfonyl)-3-nitro-1,2,4-triazole (MSNT, 3 eq) and 1-methylimidazole (2.25 eq) in DCM under dry conditions. Deprotection of the Alloc-group was done using phenyl silane as a scavenger. Coupling of 1,2,3,4-tetrahydroacridine-9-carboxylic acid was done using HATU and DIPEA in NMP. The ligand, still containing the N^{α}-Fmoc protecting group, was cleaved off the resin using TFA/water/TIS (93:5:2). The ligand was collected and purified by flash chromatography. The ligand (1 eq) was dissolved in DMF and HATU (1 eq) and DIPEA (1.2 eq) was added. The ligand was preactivated for 10 min and then added to Fractogel EMD-amino resin (1 eq, supplied by Merck KGaA and shaken for 3 h at 60° C. This procedure was then repeated twice. The Fmoc protecting group was removed using 20% piperidine in DMF for 2+30 min. The resin was washed thoroughly with DMF (5×), DCM (5×), EtOH (5×), and water (5×).

### Example 5

### Coupling of L10 to Amino Functionalized Sepharose Resin

Amino-Sepharose resin (5 ml, ˜10 μmol amino/ml) washed with 25% EtOH/water (5×), 50% EtOH/water (5×), 75% EtOH/water (5×), EtOH (5×), 25% NMP/EtOH (5×), 50% NMP/EtOH (5×), 75% NMP/EtOH (5×) and NMP (10×). hGh ligand L10 with Boc protection on the Ile amino group (3 equiv) was dissolved in NMP/DMSO (2:1, 5 ml) and EDC (3 equiv), HOAt (3 equiv) and DIPEA (4 equiv) were added. The reaction mixture stirred for 5 min and then added to the resin and shaken for 4 h at room temperature. The solvents were filtered off and the resin washed with NMP (10×) and DCM (5×).

The Boc protection was cleaved off using 30% TFA/DCM (30 min) and the resin washed with DCM (5×). The resin neutralised with 10% DIPEA/DCM (10 min) and the resin washed with DCM (5×), NMP (6×), 75% NMP/EtOH (4×), 50% NMP/EtOH (4×), 25% NMP/EtOH (4×), EtOH (6×), 75% EtOH/water (4×), 50% EtOH/water (4×), 25% EtOH/water (4×), water (6×), and 20% EtOH/water (4×).

### Example 6

### Coupling of L2 to Amino Functionalized Fractogel Resin

Ligand L2 with a Pbf protecting group on the arginine moiety and an Fmoc group on the amino group of phenylalanine (5 g) was treated with TFA/water/TIS (93:5:2) (20 ml) for 3 h at room temperature. The solvent was evaporated and the oily residue precipitated with cold diethylether. The precipitated product washed with diethylether (10×) and lyophilized yielding the product in 97% yield.

Fractogel EMD amino resin (5 ml) was washed with 25% EtOH/water (5×), 50% EtOH/water (5×), 75% EtOH/water (5×), EtOH (5×), 25% NMP/EtOH (5×), 50% NMP/EtOH (5×), 75% NMP/EtOH (5×) and NMP (10×).

Pbf cleaved ligand L2 (3 equiv) was dissolved in NMP/DMSO (2:1, 5 ml) and EDC (3 equiv), HOAt (3 equiv) and DIPEA (4 equiv) were added. The reaction mixture stirred for 5 min and added to the resin and shaken for overnight at room temperature. The solvents were filtered off and the resin washed with NMP (10×). The Fmoc was cleaved off with 20% piperidine/NMP (30 min) and the resin washed with NMP (6×), 75% NMP/EtOH (4×), 50% NMP/EtOH (4×), 25% NMP/EtOH (4×), EtOH (6×), 75% EtOH/water (4×), 50% EtOH/water (4×), 25% EtOH/water (4×), water (6×), and 20% EtOH/water (4×).

### Example 7

### Coupling of L2 to Amino Functionalized Sepharose Resin

Amino-Sepharose resin (5 ml, ˜10 μmol amino/ml) was washed with 25% EtOH/water (5×), 50% EtOH/water (5×), 75% EtOH/water (5×, EtOH (5×), 25% NMP/EtOH (5×), 50% NMP/EtOH (5×), 75% NMP/EtOH (5×) and NMP (10×). Pbf cleaved hGh ligand 006 from Example 6 (3 equiv) was dissolved in NMP/DMSO (2:1, 5 ml) and EDC (3 equiv), HOAt (3 equiv) and DIPEA (4 equiv) were added. The reaction mixture stirred for 5 min and then added to the resin and shaken for 4 h at room temperature. The solvents were filtered off and the resin washed with NMP (10×). The Fmoc was cleaved off with 20% piperidine/NMP (30 min) and the resin washed with NMP (6×), 75% NMP/EtOH (4×), 50% NMP/EtOH (4×), 25% NMP/EtOH (4×), EtOH (6×), 75% EtOH/water (4×), 50% EtOH/water (4×), 25% EtOH/water (4×), water (6×), and 20% EtOH/water (4×).

### Example 8

### Coupling of L14 to Amino Functionalized Fractogel Resin

Fractogel EMD amino resin (5 ml) was washed with 25% EtOH/water (5×), 50% EtOH/water (5×), 75% EtOH/water (5×), EtOH (5×), 25% NMP/EtOH (5×), 50% NMP/EtOH (5×), 75% NMP/EtOH (5×) and NMP (10×).

Ligand 14 with Boc protection on the alpha and epsilon amino groups of lysine (3 equiv) was dissolved in NMP/DMSO (2:1, 5 ml) and EDC (3 equiv), HOAt (3 equiv) and DIPEA (4 equiv) were. The reaction mixture was stirred for 5 min and added to the resin and shaken for overnight at room temperature. The solvents were filtered off and the resin washed with NMP (10×) then Dichloromethane (5×). The resin was then treated with 30% TFA in Dichloromethane for 30 min and the resin washed with water (5×) and EtOH (5×). The resin neutralised with 10% DIPEA/EtOH (10 min) and the resin washed with EtOH (6×), 75% EtOH/water (4×), 50% EtOH/water (4×), 25% EtOH/water (4×), water (6×), and 20% EtOH/water (4×).

### Example 9

### Coupling of L14 to Amino Functionalized Sepharose Resin

Amino-Sepharose resin (5 ml, ˜10 μmol amino/ml) was washed with 25% EtOH/water (5×), 50% EtOH/water (5×), 75% EtOH/water (5×, EtOH (5×), 25% NMP/EtOH (5×), 50% NMP/EtOH (5×), 75% NMP/EtOH (5×) and NMP (10×).

Ligand 14 with Boc protection on the alpha and epsilon amino groups of lysine (3 equiv) was dissolved in NMP/DMSO (2:1, 5 ml) and EDC (3 equiv), HOAt (3 equiv) and DIPEA (4 equiv) were added. The reaction mixture stirred for 5 min and then added to the resin and shaken for 4 h at room temperature. The solvents were filtered off and the resin washed with NMP (10×) then Dichloromethane (5×). The resin was then treated with 30% TFA in Dichloromethane for 30 min and the resin washed with water (5×) and EtOH (5×). The resin neutralised with 10% DIPEA/EtOH (10 min) and the resin washed with EtOH (6×), 75%

### Example 10

### The Binding Capacity for hGH of Ligands L2, L10 and L14 Coupled to Sepharose and Fractogel Resins

The binding capacity for wildtype hGH was measured for each resin sample in an approximately 2 mL column by
a) loading the column with a 0.5 mg/mL solution of hGH in buffer A at a flow rate of 1 mL/min,
b) washing the column with 5 or more column volumes of buffer A
c) eluting the protein by applying a gradient of 1 M NaCl/25 mM Tris at pH=7.5 (buffer B)
d) cleaning the column with 0.2 M NaOH, then with buffer B, and then with buffer A.

The binding capacity was calculated by integrating the UV signal during step (c) and indicated in the Table IV, below.

TABLE IV

| | Flow | Binding capacity |
|---|---|---|
| | rate | (pure hGH mg/ml) |
| Resin | (cm/h) | 10% breakthrough |
| L2 Fractogel | 76.5 | 13 |
| L2 Sepharose | 76.5 | 50 |
| L10-Fractogel | 76.5 | 5 |
| L10-Sepharose | 76.5 | 7 |
| L14-Fractogel | 76.5 | 8 |
| L14-Sepharose | 76.5 | 40 |

### Example 11

### Purification of Recombinant Wild Type Human Growth Hormone (hGH)

A Tricorn 5/50 (GE Healthcare Life Sciences, 1 mL) column was packed with 1.0 mL of either of the chromatographic resins prepared in Examples 3 and 4 as a 1:1 slurry in water. The column was attached to an Äkta Explorer (GE Healthcare Life Sciences) liquid chromatography system refrigerated to 16° C. The resin was washed extensively with 0.2 M NaOH until a steady UV-baseline was observed. The resin was then neutralized with 1 M NaCl, 25 mM Tris.HCl buffer (buffer B, pH=7.50) and equilibrated with 25 mM Tris.HCl buffer (buffer A, pH=7.50). Micro-filtrated E. coli cell culture fluid (2.61 mg/mL hGH titer) was filtered using a syringe driven filter (Millex HV, 0.45 mm, Millipore) and 200 μL was then diluted to 1 mL with buffer A. Total expected amount of hGH in the sample was 0.52 mg. The sample was loaded unto the column in 100% buffer A at 2 mL/min and the flow-through collected. After 10 column volumes of washing with buffer A, the adsorbed protein was eluted by applying a gradient of buffer B over 6 column volumes and the eluate collected. The column was then cleaned in place (CIP) with 0.2 M NaOH, neutralized and equilibrated for another run. The purity of the eluate was determined by SDS-PAGE using an Invitrogen Nu-PAGE system (preformed Novex 4-12% Bis-Tris gels). The gels were run using the manufacturer's protocol. Purity of the eluted protein was ca 75% for both resins.

### Example 12

Ligand L10 from Example 1 was synthesized step-wise on Fractogel Amino M (Supplied by Merck KgaA), and the resulting affinity resin was packed in Tricorn 5/50, 1 mL (GE Healthcare).

### Protein Sample:

hGH standard: hGH was dissolved to 20 mg/mL in freshly made 50 mM NH₄1-1CO₃, then diluted to 2 or 3.3 mg/mL with Buffer A (50 mM Tris-HCl, pH=7.50, T=15° C. (20° C. in Äkta)).

hGH micro-filtrate buffer: 2.61 mg/mL hGH E. coli micro-filtrate from hGH harvest. A frozen 15 mL sample was thawed at room temperature and filtered using a syringe-driven filter (0.22 mm). Diluted to 0.52 mg hGH/mL with Buffer A.

### Loading Conditions:

Buffer A1: 50 mM BisTris-HCl, pH=6.50, T=15° C. (20° C. in Äkta), flow 0.5 mL/min (150 cm/h)

### Elution Conditions:

Buffer Z1: 25 mM Tris-HCl, 1M NaCl, pH=7.50, T=15° C. (20° C. in Äkta), flow 0.5 mL/min (150 cm/h)

### Cleaning in Place (CIP) Conditions:

### 0.2 M NaOH

Dynamic 10% BT capacity (purified hGH) under these conditions was 6.7 mg/mL while dynamic 10% BT capacity for hGH micro-filtrate was 3.22 mg/mL.

An elution buffer of 200 mM Tris-HCl, pH 8.00, 1 M NaCl, 30% propylene glycol results in high recovery of hGH. This elution buffer and the aforementioned 50 mM BisTris-HCl, pH 6.5 load buffer was used. The column was overloaded with hGH. The results from an experiment using these conditions are shown below:

The loading buffer was changed to 50 mM BisTris-HCl at pH 6.25 and different elution buffers at pH 7.25, 7.50 and 8.00 were tested. The results are summarized in Table V

TABLE V
Table 5. Performance of affinity resin with ligand L10.

| | | Recovery of hGH | |
|---|---|---|---|
| Elution pH | Purity of eluate | (mg) | Recovery (%) |
| 7.25 | 91% | 1.58 | 61 |
| 7.50 | 85% | 2.08 | 81 |
| 8.00 | 85% | 2.28 | 89 |

The highest purity (91%) was observed at pH 7.25, while recovery was higher at pH 8.00 as expected. At pH 8.00 recovery was high but more impurities were present.

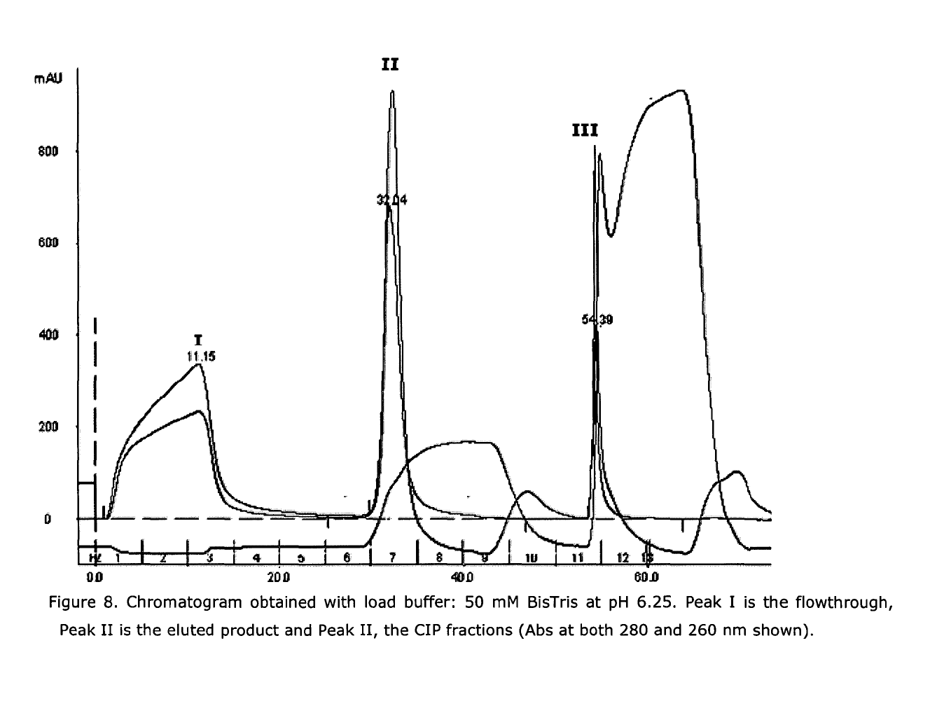

Chromatograms and gels are shown in FIG. 8 for the run with the following conditions: Load buffer: 50 mM BisTris at pH 6.25 and Elution buffer: 50 mM BisTris at pH 8.00

### Example 13

### Fractogel-Ligand L10-Free Ligand Coupling

A set of resins were prepared by coupling ligand L10 to Fractogel Amino M under three different coupling conditions. This resin has an amino density of 33 μmol/mL and was fully loaded with ligand L10.

| | | | 10% BT | | |
|---|---|---|---|---|---|
| | | | capacity | | |
| | | 10% BT | (micro- | | |
| | | capacity | filtrate | | |
| Resin name | Coupling conditions | (pure hGH) | hGH) | Purity | Recovery |
| 20700-015A | HATU/DIPEA/NMP, | 9.45 mg/mL | n/a | n/a | n/a |
| | 60° C., 5 h | | | | |
| 20700-015B | EDC/HOAt/NEM/NMP, | 8.01 mg/mL | n/a | n/a | n/a |
| | 60° C., 5 h | | | | |
| 20700-015C | EDC/HOAt/NEM/NMP, | 8.19 mg/mL | 3.38 mg/mL | 81% | 54% |
| | RT, 5 h | | | | |

All three resins behaved similarly to the resins synthesized by direct synthesis on Fractogel although with somewhat higher capacities for pure hGH. Capacity for micro-filtrate hGH was similar to the directly synthesized resin.

### Example 14

Ligand L14 from Example 1 was synthesized directly on Fractogel. The resin was evaluated in 5 mL scale.

Load buffer: 50 mM BisTris at pH 6.25

Elution buffer: 50 mM BisTris at pH 8.00 gradient

CIP: 0.2 M NaOH

Flow: 2 mL/min

Temperature: 15° C. (20° measured by Äkta)

Dynamic 10% BT capacity for pure hGH: 16.3 mg/mL

Dynamic 10% BT capacity for micro-filtrate hGH: 6.7 mg/mL

Recovery: 92%

Purity: 71%

### Example 15

Ligand L16 from Example 1 was synthesized directly on Fractogel Amino. This resin was evaluated in 5 mL scale.

Load buffer: 50 mM BisTris at pH 6.25

Elution buffer: 50 mM BisTris at pH 8.00 gradient

CIP: 0.2 M NaOH

Flow: 2 mL/min

Temperature: 20° C.

Dynamic 10% BT capacity for pure hGH: 11.9 mg/mL

Dynamic 10% BT capacity for micro-filtrate hGH: 5.7 mg/mL

Recovery: 98%

Purity: 70%

### Example 16

### Binding of Hyperglycosylated hGH to Ligands F10 and F14 on Fractogel

3 ml of cell harvest containing hyperglycosylated hGH was pH adjusted from 7.7 to 6.23 with 0.25 M HCl and diluted 3× with water.

The harvest was applied to a 1 mL column of resin equilibrated with: 50 mM BIS-TRIS pH 6.23. After application, the resin was washed (15 CV) and with the same buffer and bound protein eluted with 200 mM TrisHCl pH 8.0 (10 CV). The amount of hyperglycosylated hGH purified by both resins was 3.5 mg/mL.

### DISCUSSION

The fact that the expected high affinity ligands do bind hGH whereas the expected low affinity ligands—as expected—exhibited low affinity, indicates that the structure-fluorescence data generated in Example 1 and presented in FIG. 1 and Table 2 constitutes a good basis for predicting affinity of ligands with the general structure (IV) towards hGH.

Furthermore, it is fair to assume that the computational and experimental procedure outlined in Example 1 constitutes a good basis for designing, synthesizing, and screening large numbers of hGH affinity ligands with the general formula (I) provided that the number of combinatorial synthesis steps be adjusted according to the variables m and n, cf. formula (I).

Likewise, it is fair to assume that the experimental procedure outlined in Example 1 constitutes a good basis for screening large numbers of affinity ligands with the general formula (I) towards any protein provided that the number of combinatorial synthesis steps be adjusted according to the variables m and n, cf. formula (I), and the fluorescence screening is performed with the protein in question in place of hGH.

The synthesis and testing of novel affinity resins according to the present invention as illustrated in Examples 3-9 demonstrates the industrial applicability of said affinity resins including resin stability towards cleaning in place procedures involving exposure to NaOH solution.

SEQ ID NO: 1
FPTIPLSRLFDNAMLRAHRLHQLAFDTYQEFEEAYIPKEQKYSFLQNPQTSLCFSESIPTPSN
REETQQKSNLELLRISLLLIQSWLEPVQFLRSVFANSLVYGASDSNVYDLLKDLEEGIQTLM
GRLEDGSPRTGQIFKQTYSKFDTNSHNDDALLKNYGLLYCFRKDMDKVETFLRIVQCRSV
EGSCGF

1-19. (canceled)

20. A process for the purification of growth hormone polypeptide, said process comprising the steps of:
(a) contacting a suspension or solution containing growth hormone polypeptide with an affinity resin under conditions which facilitate binding of a portion of said growth hormone polypeptide to said affinity resin;
(b) optionally washing said affinity resin containing growth hormone polypeptide with a washing buffer; and
(c) eluting said affinity resin containing growth hormone polypeptide with an elution buffer, and collecting a Growth Hormone polypeptide as an eluate;
wherein said affinity resin is a solid phase material having covalently immobilized thereto one or more ligands of the general formula (I), 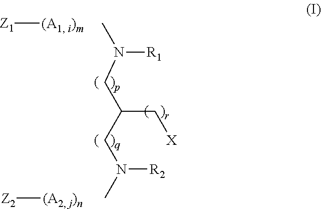
wherein
i=1, 2, . . . , m, and j=1, 2, . . . , n;
n and m are independently an integer in the range of 0-3, with the proviso that the sum n+m is in the range of 1-4;
p, q, and r are independently an integer in the range of 0-6;
A11, . . . , A1m and A21, . . . , A2n are independently selected from α-amino acid moieties, β-amino acid moieties, α-amino sulphonic acid moieties, and β-amino sulphonic acid moieties;
Z1 and Z2 are independently selected from hydrogen, C₁₋₆ alkyl, carboxylic acid moieties (Z—C(═O)—), and sulphonic acid moieties (Z—S(═O)₂—), wherein Z is selected from hydrogen, optionally substituted C₁₋₁₂-alkyl, optionally substituted C₃₋₁₂-cycloalkyl, optionally substituted C₁₋₁₂-alkenyl, optionally substituted C₁₋₁₂-alkynyl, optionally substituted aryl, optionally substituted heteroaryl and optionally substituted heterocyclyl;
R1 and R2 are independently selected from hydrogen and C₁₋₆-alkyl;
X is the group for attachment of the ligand to the solid phase material, either directly or via a linker, X being selected from carboxylic acid (—COON), a carboxylic acid ester (—COOR), a carboxylic acid anhydride (—COOCOR), a carboxylic acid halide (—COHal), sulphonic acid (—S(═O)₂OH), a sulphonyl chloride (—S(═O)₂Cl), thiol (—SH), a disulphide (—S—S—R), hydroxy (—OH), aldehyde (C(═O)H), epoxide (—CH(O)CH₂), cyanide (—CN), halogen (-Hal), primary amine (—NH₂), secondary amine (—NHR), hydrazide (—NH═NH₂), and azide (—N₃), wherein R is selected from optionally substituted C₁₋₁₂-alkyl, and Hal is a halogen; and
the total molecular weight of said ligand (excluding “X” and any linker) being 200-2000 g/mol.

21. The process according to claim 20, wherein Z1 and Z2 are independently selected from hydrogen, C₁₋₆ alkyl, xanthene-9-carbonyl, 2-amino nicotinyl, 2-quinaldincarbonyl, 4,8-dihydroxy-2-quinolinecarbonyl, 4-quinolinecarbonyl, 5-methyl-2-nitrobenzoyl, 2-(benzoimidazolylthio)acetyl, 5-methyl-2-phenyl-2H-1,2,3-triazole-4-carbonyl, 6-hydroxy-2-naphthoyl, 4,7-dimethylpyrazolo[5,1-c][1,2,4]triazine-3-carbonyl, 3-amino-4-(phenylsulfonyl)-2-thiophenecarbonyl, (+/−)-3-oxo-1-indancarbonyl, 5,6,7,8-tetrahydroacridine-9-carbonyl, 2-methylimidazo[1,2-a]pyridine-3-carbonyl, 5-(4-methyl-2-nitrophenyl)furoyl, 1-cyclohexyl-4-oxo-1,4-dihydroquinoline-3-carbonyl, quinoxaline-6-carbonyl, and 4-methyl-2-phenylpyrimidine-5-carbonyl.

22. The process according to claim 20, wherein Z1 and Z2 are independently selected from hydrogen, C₁₋₆ alkyl, xanthene-9-yl-carbonyl, 5-methyl-2-phenyl-2H-1,2,3-triazole-4-yl-carbonyl, 3-amino-(phenylsulfonyl)-thiophen-2-yl-carbonyl, (+/−)-3-oxo-1-indanyl, 5,6,7,8-tetrahydroacridine-9-yl-carbonyl, and 2-methylimidazo[1,2-a]pyridine-3-yl-carbonyl.

23. The process according to claim 20, wherein Z1 comprises a tricyclic optionally substituted heteroaromatic group.

24. The process according to claim 20, wherein said ligand has the general formula (II), 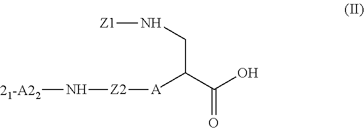
wherein
Z1 is Z—C(═O)—, wherein Z is selected from optionally substituted aryl, optionally substituted heteroaryl and optionally substituted heterocyclyl;
Z2 is selected from hydrogen, and Z—C(═O)—, wherein Z is selected from optionally substituted C₁₋₁₂-alkyl, optionally substituted C₃₋₁₂-cycloalkyl, optionally substituted aryl, optionally substituted heteroaryl and optionally substituted heterocyclyl; and
each of A2₁ and A2₂ is independently selected from α-amino acids and β-amino acids.

25. A process according to claim 24, wherein A2₁ is selected from arginine, phenylalanine, tyrosine, isoleucine, and lysine, and A2₂ is selected from arginine, phenylalanine, isoleucine, proline, tyrosine, and tryptophan.

26. A process according to claim 24 wherein said ligand has the general formula (III), 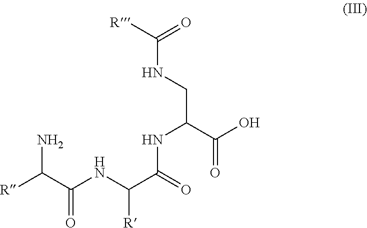
wherein R′ and R″ are independently selected from side chains of α-amino acids, and R″′ is selected from the group consisting of optionally substituted aryl, optionally substituted heteroaryl and optionally substituted heterocyclyl.

27. A process according to claim 26, wherein said ligand is selected from Nos. (1)-(16):

| No. | Structure |
|---|---|
| 1 | 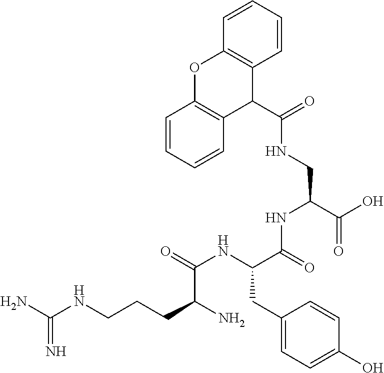 |
| 2 | 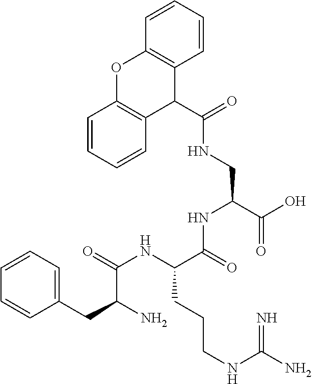 |
| 3 | 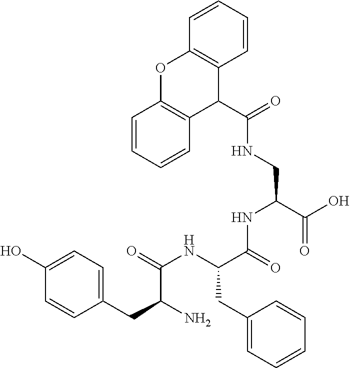 |
| 4 | 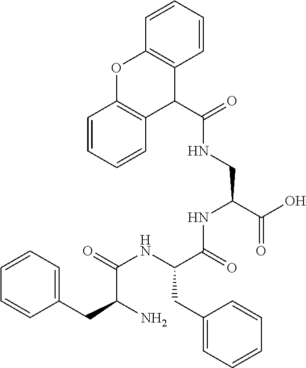 |
| 5 | 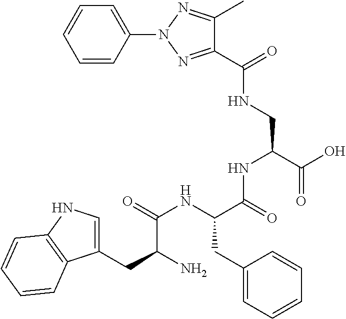 |
| 6 | 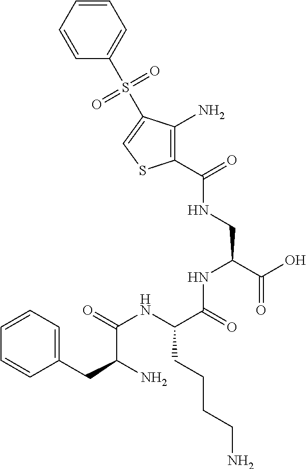 |
| 7 | 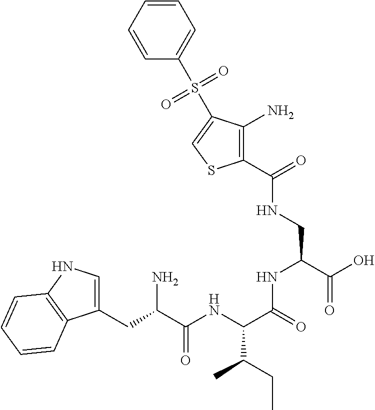 |
| 8 | 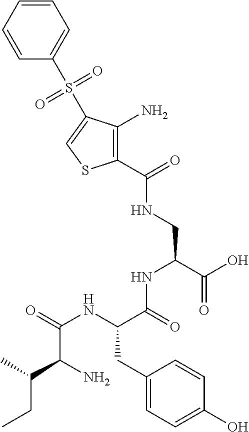 |
| 9 | 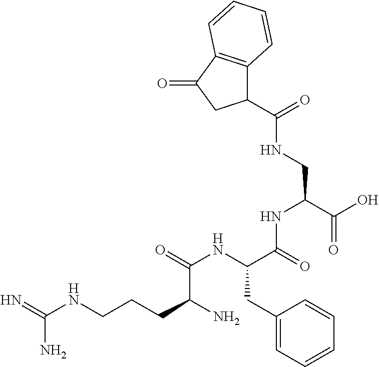 |
| 10 | 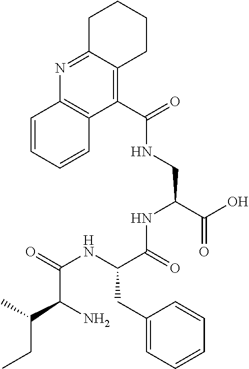 |
| 11 | 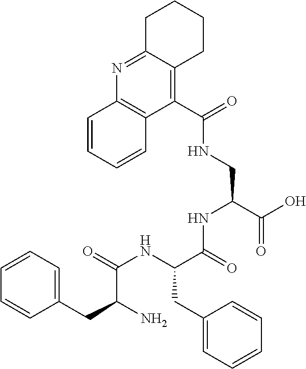 |
| 12 | 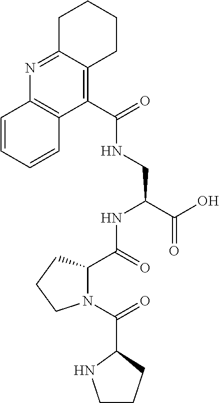 |
| 13 | 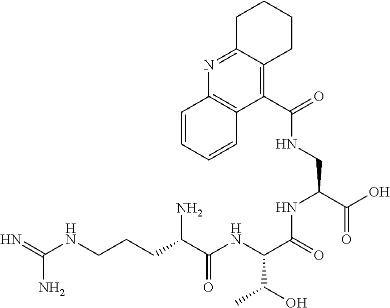 |
| 14 | 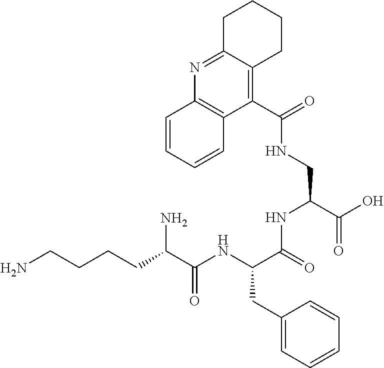 |
| 15 | 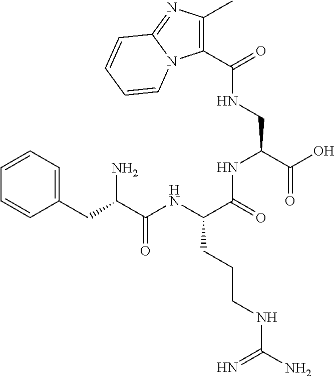 |
| 16 | 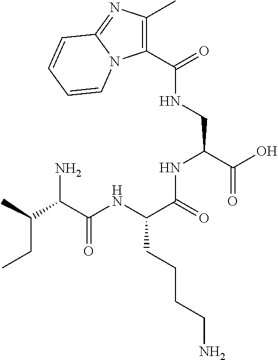 |

28. The process according to claim 20, wherein, in step (b), at least one washing buffer comprising 0-50 mM BisTris at pH 6.0-6.5.

29. The process according to claim 20, wherein, in step (c), the elution buffer has a pH between 7.0 and 8.0.

30. The process according to claim 29, wherein the elution buffer comprises 0-200 mM BisTris at pH 7.0-7.5.

31. The process according to claim 20, wherein the Growth Hormone polypeptide is a human Growth Hormone polypeptide.

32. The process according to claim 31, wherein the human Growth Hormone polypeptide is a recombinant human Growth Hormone polypeptide.

33. The process according to claim 31, wherein the human Growth Hormone polypeptide is a modified human Growth Hormone polypeptide.

34. The process according to claim 32, wherein the human recombinant Growth Hormone polypeptide is a modified recombinant human Growth Hormone polypeptide.

35. The process according to claim 33, wherein the modified human Growth Hormone polypeptide is a PEGylated human Growth Hormone polypeptide.

36. The process according to claim 34, wherein the modified recombinant human Growth Hormone polypeptide is a PEGylated recombinant human Growth Hormone polypeptide.

37. An affinity resin comprising a solid phase material having covalently immobilized thereto one or more ligands of the formula (I) 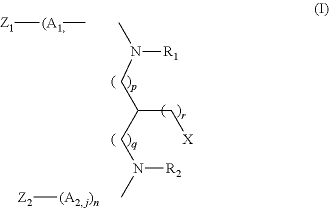
wherein
i=1, 2, . . . , m, and j=1, 2, . . . , n;
n and m are independently an integer in the range of 0-3, with the proviso that the sum n+m is in the range of 1-4;
p, q, and r are independently an integer in the range of 0-6;
A11, . . . , A1m and A21, . . . , A2n are independently selected from α-amino acid moieties, β-amino acid moieties, α-amino sulphonic acid moieties, and β-amino sulphonic acid moieties;
Z1 and Z2 are independently selected from hydrogen, C₁₋₆ alkyl, carboxylic acid moieties (Z—C(═O)—), and sulphonic acid moieties (Z—S(═O)₂—), wherein Z is selected from hydrogen, optionally substituted C₁₋₁₂-alkyl, optionally substituted C₃₋₁₂-cycloalkyl, optionally substituted C₁₋₁₂-alkenyl, optionally substituted C₁₋₁₂-alkynyl, optionally substituted aryl, optionally substituted heteroaryl and optionally substituted heterocyclyl;
R1 and R2 are independently selected from hydrogen and C₁₋₆-alkyl;
X is the group for attachment of the ligand to the solid phase material, either directly or via a linker, X being selected from carboxylic acid (—COOH), a carboxylic acid ester (—COOR), a carboxylic acid anhydride (—COOCOR), a carboxylic acid halide (—COHal), sulphonic acid (—S(═O)₂OH), a sulphonyl chloride (—S(═O)₂Cl), thiol (—SH), a disulphide (—S—S—R), hydroxy (—OH), aldehyde (C(═O)H), epoxide (—CH(O)CH₂), cyanide (—CN), halogen (-Hal), primary amine (—NH₂), secondary amine (—NHR), hydrazide (—NH═NH₂), and azide (—N₃), wherein R is selected from optionally substituted C₁₋₃₂-alkyl, and Hal is a halogen; and the total molecular weight of said ligand (excluding “X” and any linker) being 200-2000 g/mol.

38. The affinity resin according to claim 37, wherein the ligand is as specified in claim 2.

39. The affinity resin according to claim 38, wherein the ligand is selected from the group consisting of ligands (1)-(16) as defined in claim 27, wherein the ligand is covalently attached to the solid phase material of said affinity resins via the carboxylic acid group, either directly or via a linker.

40. An affinity ligand selected from the group consisting of ligands (1)-(16) defined herein.
